(19) United States
(12) Patent Application Publication
(10) Pub. No.: US 2026/0199343 A1
(43) Pub. Date: Jul. 16, 2026

(54) Methods of Administering Belumosudil for Treatment of Multiple Myeloma

(71) Applicant: Kadmon Corporation, LLC, Morristown, NJ (US)

(72) Inventors: Kamlesh Bisht, Cambridge, MA (US); Monsif Bouaboula, Cambrige, MA (US); Marielle Chiron, Paris (FR); Marco Meloni, Paris (FR); Helgi Van De Velde, Diegem (BE); Angela Virone-Oddos, Paris (FR)

(73) Assignee: Kadmon Corporation, LLC, Morristown, NJ (US)

(21) Appl. No.: 19/133,522

(22) PCT Filed: Dec. 8, 2023
(86) PCT No.: PCT/US2023/083039

§ 371 Date: May 28, 2025

Related U.S. Application Data
(60) Provisional application No. 63/431,446, filed on Dec. 9, 2022.

(30) Foreign Application Priority Data
Jan. 26, 2023 (EP) 23305096.2

(51) Int. Cl. A61K 31/517; A61K 31/454; A61K 31/573; A61K 39/00; A61P 35/00; C07K 16/28

(52) U.S. Cl. CPC A61K 31/517; A61K 31/454; A61K 31/573; A61P 35/00; C07K 16/2896; A61K 2039/545; C07K 2317/24; C07K 2317/732; C07K 2317/734

(57) ABSTRACT

The present disclosure provides methods of administering belumosudil to patients with multiple myeloma.

### TECHNICAL FIELD

The present disclosure relates to methods of administering belumosudil to patients for the treatment of multiple myeloma.

### BACKGROUND

Multiple myeloma (MM) is a malignant neoplasm of the plasma cells that accumulate in bone marrow, leading to renal failure, hypercalcemia, bone destruction, and anemia due to marrow failure. MM accounts for approximately 1.8% of all cancers and 18.7% of hematologic malignancies in the United States. (Siegel et al. C A Cancer J Clin 2021, 71:7-33). MM is most frequently diagnosed among people aged 65 to 74 years, with the median age being 69 years. (National Cancer Institute. Surveillance, Epidemiology, and End Results Program. Cancer Stat Facts: Myeloma. Accessed Nov. 24, 2021. Available at: https://seer.cancer.gov/statfacts/html/mulmy.html). The American Cancer Society has estimated 34,920 new MM cases will be diagnosed in the United States in 2021, with an estimated 12,410 deaths. (Siegel et al.). There is a significant need for additional therapies to treat multiple myeloma.

CD38 expression level on MM cells is a critical factor that affect the response to anti-CD38 antibodies like isatuximab and daratumumab. (Kitadate et al. Haematologica. 2020, 105(1):e37-e40). Upon binding to CD38-expressing MM cells, anti-CD38 antibodies like isatuximab and daratumumab induce tumor cell killing via antibody dependent cellular cytotoxicity (ADCC), antibody-dependent cellular phagocytosis (ADCP), and complement-dependent cytotoxicity (CDC), as well as via direct apoptosis. (Zhu et al. Front Immunol. 2020; 11, 1771).

Several studies reported that CD38 expression is reduced in the bone marrow and MM cells of patients after first treatment with daratumumab. (Horenstein et al. Cells. 2015, 4(3), 520-37; Krejcik et al. Clin Cancer Res. 2017, 23(24), 7498-511; Nijhof et al. Blood. 2016, 128(7), 959-70), and that CD38 expression increases again after daratumumab discontinuation (Nijhof et al.). Moreover, isatuximab reduces CD38 expression in several MM cell lines by inducing CD38 internalization (Moreno et al. Clin Cancer Res. 2019, 25(10), 3176-87).

In vitro studies using well characterized human MM cells lines are recognized as adequate models. See, e.g., Kassem et al, Blood 2022 Feb. 24, 139(8):1160-1176 (“Kassem”); Matsuo et al, Leuk Res 2004 August, 28(8):869-77; and Deckert et al, Clin Cancer Res 2014 Sep. 1, 20(17):4574-83, describing MOLP-8 MM cell lines, Kassem; De Veirman K et al, Oncotarget 2015, 6:10532-10547 (“De Veirman”); Li M et al, Cancer Cell Int 2021 Dec. 19, 21(1):683 (“Li”); and Moreaux J et al, Haematologica, 2011 April, 96(4):574-82 (“Moreaux”), describing RPMI-8226 MM cell lines, Moreaux; De Veirman; and Li describing LP-1 MM cell lines, and Chauhan D et al, Blood 2004 Apr. 15, 103(8):3158-66; and Yasui H et al, Blood 2005 Jul. 15, 106(2):706-12, describing MM.1R cell lines.

The present disclosure provides one solution to the problem of multiple myeloma by providing methods of treating multiple myeloma with belumosudil alone as well as in combination with antimyeloma agents such as anti-CD38 antibodies or immunomodulatory drugs (IMiDs).

### SUMMARY

The present disclosure relates to methods of treating multiple myeloma comprising administering to a subject in need thereof a therapeutically effective amount of 2-{3-[4-(1H-indazol-5-ylamino)-2-quinazolinyl]phenoxy}-N-(propan-2-yl) acetamide, or a pharmaceutically acceptable salt thereof (belumosudil).

The present disclosure also relates to methods of treating multiple myeloma comprising administering to a subject in need thereof a therapeutically effective amount of 2-{3-[4-(1H-indazol-5-ylamino)-2-quinazolinyl]phenoxy}-N-(propan-2-yl) acetamide, or a pharmaceutically acceptable salt thereof (belumosudil) and an anti-CD38 antibody.

The present disclosure also relates to methods of treating multiple myeloma comprising administering to a subject in need thereof a therapeutically effective amount of 2-{3-[4-(1H-indazol-5-ylamino)-2-quinazolinyl]phenoxy}-N-(propan-2-yl) acetamide, or a pharmaceutically acceptable salt thereof (belumosudil) and an immunomodulatory drug (IMiD).

The present disclosure also relates to methods of overcoming IMiD resistance in multiple myeloma subjects comprising administering to a subject in need thereof a therapeutically effective amount of 2-{3-[4-(TH-indazol-5-ylamino)-2-quinazolinyl]phenoxy}-N-(propan-2-yl) acetamide, or a pharmaceutically acceptable salt thereof (belumosudil), optionally with an anti-CD38 antibody or an immunomodulatory drug (IMiD).

The present embodiments can be understood more fully by reference to the detailed description and examples, which are intended to exemplify non-limiting embodiments.

### BRIEF DESCRIPTION OF THE DRAWINGS

FIGS. 1A and 1B show the effect of treatment with either belumosudil or pomalidomide on the proliferation of multiple myeloma cell lines.

FIG. 2 shows a Western blot of C-Myc and b-Actin after 24 hours of treatment of LP1 cells with either belumosudil or pomalidomide.

FIGS. 3A, 3B, 3C, and 3D show flow cytometry data. Data were analyzed with FlowJo software using the following strategy: (A) MOLP-8 cells excluding debris were gated, followed by gating for single cells (B), from single cells, viable DAPI^{neg} cells were quantified (C), and from DAPI^{neg} cells, the expression of CD38 was quantified with FITC fluorochrome (D) and expressed as median of fluorescence intensity (MFI).

FIG. 4 shows CD38 expression on MOLP-8 cells after 3 or 4 days of treatment with isotype control (IC) plus belumosudil, isatuximab plus belumosudil, or belumosudil alone as compared to vehicle control (DMSO).

FIGS. 5A, 5B, 5C, 5D, and 5E show the viability of MOLP-8 cells after treatment with isotype control (IC) plus belumosudil, isatuximab plus belumosudil, or belumosudil alone as compared to vehicle control (DMSO).

FIGS. 6A, 6B, and 6C show the viability of LP1, RPM18226, and healthy donor (HD)-derived NK cells after treatment with belumosudil as compared to vehicle control (DMSO).

FIGS. 7A, 7B, and 7C show flow cytometry data. Data were analyzed with FlowJo software using the following strategy in FIGS. 7A-7C: MOLP-8 cells excluding debris were gated (A), followed by gating for single cells (B). From single cells, early apoptotic 7-AAD^{neg} and Annexin V-eFluor450^{pos} (C, Q 1) and late apoptotic 7-AAD^{pos} and Annexin V-eFluor450^{pos} (C, Q2) cells were quantified (C).

FIG. 8 shows the percent of apoptotic MOLP-8 MM cells 2, 3, and 4 days after treatment with isotype control (IC) plus belumosudil, isatuximab plus belumosudil, or belumosudil alone as compared to vehicle control (DMSO).

FIG. 9 shows cytotoxicity of LP-1 RFP cells with or without HD NK cells after treatment with belumosudil.

FIG. 10 shows cytotoxicity of LP-1 RFP cells with HD NK cells after treatment with isotype control (IC, 100 ng/ml), isatuximab (100 ng/ml), belumosudil (3.3 M), IC plus belumosudil (100 ng/ml and 3.3 μM, respectively), or isatuximab plus belumosudil (100 ng/ml and 3.3 μM, respectively).

FIGS. 11A-11G shows graphs of cell viability, expressed as either number of cells or percentage of live cells, of MOLP-8 (FIGS. 11A and 11E), LP-1 (FIGS. 11C and 11G), and RPMI-8226 (FIGS. 11B and 11F) multiple myeloma cell lines, and of HD NK cells (FIG. 11D), after treatment with various concentrations of belumosudil and Y27632.

FIG. 12 shows cytotoxicity of RPMI-8226 RFP cells with HD NK cells after treatment with isotype control (IC, 10 ng/ml), isatuximab (10 ng/ml), belumosudil (1.1 or 3.3 μM), IC plus belumosudil (10 ng/ml and 1.1 or 3.3 μM, respectively), or isatuximab plus belumosudil (10 ng/ml and 1.1 or 3.3 μM, respectively).

FIG. 13 shows cytotoxicity of MM.1R RFP cells with HD NK cells after treatment with isotype control (IC, 10 ng/ml), isatuximab (10 ng/ml), belumosudil (1.1 or 3.3 μM), IC plus belumosudil (10 ng/ml and 1.1 or 3.3 μM, respectively), or isatuximab plus belumosudil (10 ng/ml and 1.1 or 3.3p M, respectively).

FIG. 14 shows cytotoxicity of LP-1 RFP cells with HD NK cells after treatment with isotype control (IC, 100 ng/ml), isatuximab (100 ng/ml), belumosudil (1.1 or 3.3 μM), IC plus belumosudil (100 ng/ml and 1.1 or 3.3 μM, respectively), or isatuximab plus belumosudil (100 ng/ml and 1.1 or 3.3 μM, respectively).

FIGS. 15A and 15B show growth inhibition activity of belumosudil (Rezurock) in MM1S cells (FIG. 15A) and in LP-1 cells (FIG. 15B), as a single agent.

FIGS. 16A-16C shows growth inhibition activity of the combination of belumosudil and dexamethasone in MM1S cells. FIG. 16A is a heat map showing inhibition of cell growth at various concentrations of belumosudil and dexamethasone; FIG. 16B is a three-dimensional plot of the data in FIG. 16A showing synergy on the Z axis; and FIG. 16C is a plot of percent confluence over time for the single agents and the combination.

FIGS. 17A-17C shows growth inhibition activity of the combination of belumosudil and dexamethasone in LP-1 cells. FIG. 17A is a heat map showing inhibition of cell growth at various concentrations of belumosudil and dexamethasone; FIG. 17B is a three-dimensional plot of the data in FIG. 17A showing synergy on the Z axis; and FIG. 17C is a plot of percent confluence over time for the single agents and the combination.

### DETAILED DESCRIPTION

### Overview

Belumosudil is an oral selective rho-associated coiled-coil-containing protein kinase-2 (ROCK2) inhibitor. ROCK2 inhibition acts on the dysregulated adaptive immune system and the fibrosis that occurs because of aberrant tissue repair. Belumosudil inhibits ROCK2 and ROCK1 with IC₅₀ values of approximately 100 nM and 3 μM, respectively.

Belumosudil down-regulated proinflammatory responses via regulation of STAT3/STAT5 phosphorylation and shifting Th17/Treg balance in ex-vivo or in vitro-human T cell assays. Belumosudil also inhibited aberrant pro-fibrotic signaling, in vitro. By controlling ROCK2 activity, belumosudil mediates signaling in immune cellular function and fibrotic pathways, thereby alleviating the effects caused by this debilitating disease, such as inflammation of multiple tissues and fibrotic changes that may involve several organs including the lungs, hepatobiliary system, musculoskeletal system, gastrointestinal (GI) tract, and skin. In vivo, belumosudil demonstrated activity in animal models of chronic GVHD.

The mesylate salt of belumosudil is marketed as REZUROCK™ in the United States and other countries for the treatment of patients with chronic GVHD (cGVHD), in some instances after failure of at least two prior lines of systemic therapy. The compound belumosudil has the chemical name: 2-{3-[4-(1H-indazol-5-ylamino)-2-quinazolinyl]phenoxy}-N-(propan-2-yl) acetamide. The compound belumosudil is also known as KD025. The active pharmaceutical ingredient of REZUROCK™ is belumosudil mesylate salt with the molecular formula C₂₇H₂₈N₆O₅S, a molecular weight of 548.62 g/mol, and having the chemical name 2-{3-[4-(1H-indazol-5-ylamino)-2-quinazolinyl]phenoxy}-N-(propan-2-yl) acetamide methanesulfonate (1:1).

The chemical structure of belumosudil mesylate is as follows:

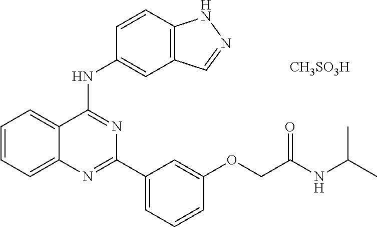

Belumosudil and processes for making the compound are described in the following US patents: U.S. Pat. Nos. 8,357,693, 9,815,820, 10,183,931, and 10,696,660.

The present disclosure provides methods of administering belumosudil mesylate (REZUROCK™) to patients in need thereof for the treatment of multiple myeloma. Also disclosed are methods of administering belumosudil mesylate (REZUROCK™) in combination with antimyeloma agents such as anti-CD38 antibodies or immunomodulatory drugs (IMiDs) to patients in need thereof for the treatment of multiple myeloma.

### Definitions

“About” as used herein includes the exact amount modified by the term, about, as well as an amount that would be expected to be within experimental error, such as for example, within 15%, 10%, or 5%. For example, “about 200 mg” means “200 mg” and also a range of mgs that is within experimental error, e.g., plus or minus 15%, 10%, or 5% of 200 mg. As used herein, the term “about” may be used to modify a range and also, a particular value.

“Administering” or “administered to” as used herein (for example, including use of this term with reference to cease administration, and/or resuming administration, of API, including Compound or belumosudil, to a subject), refers to the act of prescribing medicine(s) containing the API for the subject to take during treatment, the act of dispensing the medicine(s) to the subject, and/or the act of physically receiving or ingesting the medicine(s). Thus, the API (e.g., Compound or belumosudil), can be “administered” by a physician or other medical professional who writes prescriptions for medicine(s); and/or by a pharmacist who fills said prescriptions and/or dispenses the medicine(s) to the subject; and/or by the patient or subject who ingests the medicine and/or his or her partner or caretaker who provides the medicine to a subject, each of whom also may “cease” administration and/or “resume” administration of the API.

When the term “belumosudil” is used herein, it should be understood that, unless the context clearly indicates otherwise, the term may cover the compound belumosudil in any form as well as pharmaceutically acceptable salts thereof. The term “belumosudil” refers both to the compound belumosudil (for example, in the free base form, amorphous form, or crystalline form), to pharmaceutically acceptable salts of belumosudil, for example, the mesylate salt form as used in REZUROCK™ and to any form of belumosudil that may be used in a formulation or pharmaceutical composition for administering the compound to a patient.

“Line of treatment” or “line of therapy” describes the sequence or order in which different therapies are given to a patient as the patient's disease progresses. Initial treatment (first-line therapy) may not work or may stop working after a period. After first-line therapy is discontinued, a second different treatment (second-line therapy) may be given. Subsequent lines of therapy may be given when a second-line therapy does not work or stops working. Some patients may be administered multiple lines of therapy over the course of a disease.

“Or” is used in the inclusive sense (equivalent to “and/or”) unless the context requires otherwise.

“Patient” or “subject” as used herein includes an animal or a human, in one embodiment, a human.

“Pharmaceutical composition” means a mixture of substances suitable for administering to an individual that includes a pharmaceutical agent. For example, a pharmaceutical composition may comprise a sterile aqueous solution or the API formulated in an oral dosage form such as a tablet or capsule.

“Pharmaceutically acceptable salt” means a physiologically and pharmaceutically acceptable salt of a compound provided herein. A “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred.

“Relapse” or “relapsed” means the reappearance of signs and symptoms of a disease or condition after a period of improvement. In multiple myeloma, relapse involves one or more of the following: (1) definite increase in the size of existing plasmacytomas or bone lesions, (2) hypercalcemia, which is above normal calcium levels in the blood (>11 mg/dL), (3) decrease in hemoglobin (protein in red blood cells that carries oxygen) of ≥2 g/dL not related to therapy or other non-myeloma-related conditions, (4) rise in serum creatinine (muscle waste product in the blood) by 2 mg/dL or more from the start of the therapy and attributable to myeloma, (5) hyperviscosity (thickening of the blood) related to serum protein, (6) increase of 25% from the lowest confirmed response value in one or more of the following criteria: a) serum M-protein (the increase must be at least 0.5 g/dL), b) urine M-protein (the increase must be at least 200 mg/24 hours), c) if no serum or urine M-protein can be measured, the difference between the involved (abnormal, or monoclonal) and uninvolved (normal or polyclonal) free light chain levels (the increase must be >10 mg/dL). (Kumar et al. Lancet Oncol, 2016, 17(8), E328-E346).

“Refractory” means a disease or condition that is initially unresponsive to treatment, or becomes unresponsive to treatment over time.

A “therapeutically effective amount” of an active pharmaceutical ingredient (API) means an amount which, when administered to a human for treating a disease (for example, multiple myeloma), is sufficient to effect treatment for the disease state being treated. As applied to multiple myeloma in a human, “treating” or “treatment” includes (1) reducing the risk of developing multiple myeloma and/or inhibiting multiple myeloma, i.e., arresting or reducing the development of multiple myeloma or its clinical symptoms; and (2) relieving multiple myeloma, i.e., causing regression, reversal, or amelioration of the multiple myeloma or reducing the number, frequency, duration or severity of its clinical symptoms. The therapeutically effective amount of an API may vary depending upon the health and physical condition of the subject to be treated, the extent of disease progression, the assessment of the medical situation, and other relevant factors.

### EXEMPLARY EMBODIMENTS

In one embodiment, the present disclosure provides for a method of treating multiple myeloma, comprising administering to a subject in need thereof a therapeutically effective amount of 2-{3-[4-(1H-indazol-5-ylamino)-2-quinazolinyl]phenoxy}-N-(propan-2-yl) acetamide, or a pharmaceutically acceptable salt thereof (belumosudil).

In some embodiments, the belumosudil is administered as a monotherapy. In some embodiments, belumosudil is administered in combination with an anti-CD38 antibody.

In some embodiments, the anti-CD38 antibody is daratumumab, isatuximab, MOR202, or mezagitamab (TAK-079). In some embodiments, the anti-CD38 antibody is isatuximab. In some embodiments, the isatuximab is administered to the subject intravenously at a dose of 10 mg/kg or 20 mg/kg. In some embodiments, the isatuximab is administered subcutaneously at a dose of 1000 mg or 1400 mg. In some embodiments, the isatuximab is administered in a 28-day cycle. In some embodiments, in a first 28-day cycle the isatuximab is administered once per week. In some embodiments, in subsequent 28-day cycles the isatuximab is administered twice per week. In some embodiments, after 12 months of isatuximab administration, the isatuximab is administered once every 4 weeks.

In some embodiments, belumosudil is administered in combination with an immunomodulatory drug (IMiD).

In some embodiments, the IMiD is selected from pomalidomide, lenalidomide, thalidomide, iberdomide, and mezigdomide. In some embodiments, the IMiD is pomalidomide, and the pomalidomide is administered to the subject at a dose of 4 mg once per day from day 1 to day 21 of each 28-day cycle. In some embodiments, the pomalidomide is administered orally.

In some embodiments, the belumosudil is administered in a 28-day cycle. In some embodiments, the belumosudil is administered to the subject at a dose of up to 1000 mg per day. In some embodiments, the belumosudil is administered to the subject at a dose of 200 mg per day. In some embodiments, the belumosudil is administered to the subject at a dose of 200 mg twice per day. In some embodiments, the belumosudil is administered to the subject at a dose of 400 mg per day.

In some embodiments, the belumosudil is administered to the subject at a dose of 500 mg per day, 600 mg per day, 700 mg per day, 800 mg per day, 900 mg per day and 1000 mg per day. In some embodiments, the belumosudil is administered orally.

In some embodiments, belumosudil is the mesylate salt of 2-{3-[4-(1H-indazol-5-ylamino)-2-quinazolinyl]phenoxy}-N-(propan-2-yl) acetamide.

In some embodiments, the subject is receiving concomitant corticosteroid therapy. In some embodiments, the concomitant corticosteroid therapy is selected from dexamethasone, prednisone, and methylprednisolone.

In some embodiments, the method of treating multiple myeloma, comprising administering to a subject in need thereof a therapeutically effective amount of 2-{3-[4-(1H-indazol-5-ylamino)-2-quinazolinyl]phenoxy}-N-(propan-2-yl) acetamide, or a pharmaceutically acceptable salt thereof (belumosudil) further comprises administering isatuximab and dexamethasone.

In some embodiments, the method of treating multiple myeloma, comprising administering to a subject in need thereof a therapeutically effective amount of 2-{3-[4-(1H-indazol-5-ylamino)-2-quinazolinyl]phenoxy}-N-(propan-2-yl) acetamide, or a pharmaceutically acceptable salt thereof (belumosudil) further comprises administering pomalidomide and dexamethasone.

In some embodiments, the subject has received prior therapy for treating the multiple myeloma. In some embodiments, the subject has relapsed multiple myeloma. In some embodiments, the subject has relapsed and refractory multiple myeloma. In some embodiments, the multiple myeloma is smoldering multiple myeloma.

In some embodiments, the treatment overcomes IMiD resistance in the subject with multiple myeloma.

In some embodiments, use of a therapeutically effective amount of 2-{3-[4-(1H-indazol-5-ylamino)-2-quinazolinyl]phenoxy}-N-(propan-2-yl) acetamide, or a pharmaceutically acceptable salt thereof (belumosudil) for preparing a medicament for the treatment of multiple myeloma in a subject in need thereof is provided.

In some embodiments, a compound comprising a therapeutically effective amount of 2-{3-[4-(1H-indazol-5-ylamino)-2-quinazolinyl]phenoxy}-N-(propan-2-yl) acetamide, or a pharmaceutically acceptable salt thereof (belumosudil) for use in the treatment of multiple myeloma in a subject in need thereof is provided.

### Administration of Belumosudil

In some embodiments, the belumosudil is administered to the subject at a dose of about 1000 mg per day, 900 mg per day, 800 mg per day, 700 mg per day, 600 mg per day, or 500 mg per day. In some embodiments, the belumosudil is administered to the subject at a dose selected from 200 mg per day, 200 mg twice per day, and 400 mg per day. In some embodiments, the dose is 200 mg per day. In some embodiments, the dose is 200 mg twice per day. In some embodiments, the dose is 400 mg per day.

In some embodiments, the belumosudil is administered in a 28-day cycle.

### Belumosudil Tablets

In one embodiment, the belumosudil is formulated into a tablet for oral administration. Belumosudil mesylate is a yellow powder that is practically insoluble in water. Belumosudil tablets may be prepared for oral administration. Each tablet contains 200 mg of the free base equivalent to 242.5 mg of belumosudil mesylate. The tablet also may contain the following inactive ingredients: microcrystalline cellulose, hypromellose, croscarmellose sodium, colloidal silicon dioxide, and magnesium stearate. The tablet film consists of polyvinyl alcohol, polyethylene glycol, talc, titanium dioxide and yellow iron oxide. Each 200 mg tablet is a pale-yellow film-coated oblong tablet debossed with “KDM” on one side and “200” on the other side. Tablets are stored at room temperature, 20° C. to 25° C. (68° F. to 77° F.); excursions permitted from 15° C. and 30° C. (59° F. to 86° F.).

### Multiple Myeloma

In vitro studies using well characterized MM cells lines are recognized as adequate models. Thus, studies in the MM cell lines MOLP-8, LP-1, RPMI-8226, and MM.1R are understood to be relevant in vitro models for assessing multiple myeloma therapeutic agents. See, e.g., Kassem S et al, Blood 2022 Feb. 24; 139(8):1160-1176; Matsuo Y et al, Leuk Res. 2004 August; 28(8):869-77; Deckert J et al, Clin Cancer Res. 2014 Sep. 1; 20(17):4574-83; De Veirman K et al, Oncotarget. 2015; 6:10532-10547; Li M et al, Cancer Cell Int. 2021 Dec. 19; 21(1):683; Moreaux J et al, Haematologica. 2011 April; 96(4):574-82; Chauhan D et al, Blood 2004 Apr. 15; 103(8):3158-66; and Yasui H et al, Blood 2005 Jul. 15; 106(2):706-12.

The Revised International Myeloma Working Group diagnostic criteria for multiple myeloma requires clonal bone marrow plasma cells >10% or biopsy-proven bony or extramedullary plasmacytoma and any one or more of the following myeloma defining events: (1) evidence of end organ damage that can be attributed to the underlying plasma cell proliferative disorder, specifically: (a) hypercalcaemia: serum calcium >0.25 mmol/L (>1 mg/dL) higher than the upper limit of normal or >2.75 mmol/L (>11 mg/dL); (b) renal insufficiency: creatinine clearance <40 mL per min (measured or estimated by validated equations) or serum creatinine >177 μmol/L (>2 mg/dL); (c) anaemia: haemoglobin value of >20 g/L below the lower limit of normal, or a haemoglobin value <100 g/L; or (d) bone lesions: one or more osteolytic lesions on skeletal radiography, CT, or PET-CT (If bone marrow has less than 10% clonal plasma cells, more than one bone lesion is required to distinguish from solitary plasmacytoma with minimal marrow involvement); (2) any one or more of the following biomarkers of malignancy: (a) clonal bone marrow plasma cell percentage >60%; (b) involved:uninvolved serum free light chain ratio >100 (these values are based on the serum Freelite assay (The Binding Site Group, Birmingham, UK), the involved free light chain must be ≥100 mg/L); or (c) >1 focal lesions on MRI studies (Each focal lesion must be 5 mm or more in size). (Rajkumar et al. Lancet Oncol. 2014, 15(12), e538-48).

The Revised International Myeloma Working Group diagnostic criteria for smouldering multiple myeloma require both of the following criteria: (1) Serum monoclonal protein (IgG or IgA) ≥30 g/L or urinary monoclonal protein ≥500 mg per 24 h and/or clonal bone marrow plasma cells 10-60%; and (2) absence of myeloma defining events or amyloidosis. (Rajkumar et al.).

In one embodiment, PET-CT=¹⁸F-fluorodeoxyglucose PET with CT should be measured. Clonality should be established by showing κ/λ-light-chain restriction on flow cytometry, immunohistochemistry, or immunofluorescence. Bone marrow plasma cell percentage should preferably be estimated from a core biopsy specimen; in case of a disparity between the aspirate and core biopsy, the highest value should be used.

CRAB is the acronym for the most common symptoms of multiple myeloma: hypercalcaemia, renal failure, anaemia, and bone lesions.

Multiple myeloma may be classified into different types based on the immunoglobulin that is overproduced by the myeloma cell. The majority of myeloma patients have immunoglobulin G (IgG) or immunoglobulin A (IgA) myeloma. Rare subtypes are immunoglobulin D (IgD) and immunoglobulin E (IgE) myeloma. Approximately 15% to 20% of patients have light chain myeloma (Bence Jones myeloma). About 1% to 5% of patients have non-secretory myeloma. IgM myeloma is a very rare subtype, which typically occurs in Waldenstrom's macroglobulinemia.

Drugs used to treat multiple myeloma, include proteasome inhibitors (PIs), immunomodulatory drugs (IMiDs), monoclonal antibodies (MAbs), and corticosteroids. Bisphosphonates may also be used to manage myeloma-related bone disease.

Initial treatment in patients eligible for transplantation may include induction or first-line therapy prior to autologous stem cell transplantation (ASCT). Induction therapies may include a combination of bortezomib, lenalidomide and dexamethasone (VRd); carfilzomib, lenalidomide and dexamethasone; daratumumab, lenalidomide, bortezomib and dexamethasone; ixazomib, lenalidomide and dexamethasone; bortezomib, cyclophosphamide and dexamethasone (VCD or CyBorD); bortezomib, doxorubicin and dexamethasone; carfilzomib, cyclophosphamide and dexamethasone; ixazomib, cyclophosphamide and dexamethasone; bortezomib, thalidomide and dexamethasone (VTD); cyclophosphamide, lenalidomide and dexamethasone; lenalidomide and dexamethasone (RD); bortezomib and dexamethasone (VD); daratumumab and hyaluronidase; bortezomib, melphalan and prednisone; daratumumab and hyaluronidase, lenalidomide and dexamethasone; daratumumab and hyaluronidase, bortezomib, thalidomide and dexamethasone; daratumumab, carfilzomib, lenalidomide and dexamethasone; daratumumab, cyclophosphamide, bortezomib and dexamethasone; daratumumab, bortezomib, thalidomide and dexamethasone; or dexamethasone, thalidomide, cisplatin, doxorubicin, cyclophosphamide, etoposide and bortezomib.

After maximum response to induction therapy has been achieved, an autologous stem cell transplant (ASCT) followed by maintenance therapy may be used. Maintenance therapy may include lenalidomide; ixazomib; bortezomib; or bortezomib and lenalidomide with or without dexamethasone.

Initial treatment in patients not eligible for transplantation may include bortezomib, lenalidomide and dexamethasone; daratumumab, lenalidomide and dexamethasone; ixazomib, lenalidomide and dexamethasone; daratumumab, bortezomib, melphalan and prednisone; or daratumumab, cyclophosphamide, bortezomib and dexamethasone. Maintenance therapy for patients not eligible for transplantation may include lenalidomide, ixazomib, bortezomib, or bortezomib and lenalidomide.

Therapy for treatment of relapsed multiple myeloma may include bortezomib, lenalidomide and dexamethasone; carfilzomib, lenalidomide and dexamethasone; daratumumab, bortezomib and dexamethasone, daratumumab, carfilzomib and dexamethasone; daratumumab, lenalidomide and dexamethasone; ixazomib, lenalidomide and dexamethasone; or isatuximab, carfilzomib and dexamethasone. Treatments after two prior multiple myeloma therapies may include ixazomib, pomalidomide and dexamethasone; pomalidomide, bortezomib and dexamethasone; isatuximab, pomalidomide and dexamethasone; or daratumumab, pomalidomide and dexamethasone.

Smoldering multiple myeloma (SMM) is a precancerous form of multiple myeloma that typically accounts for about 15% of newly diagnosed multiple myeloma cases. It is diagnosed when low levels of M protein are found in the blood and a slightly increased number of plasma cells are found in the bone marrow. Many patients with SMM are asymptomatic, but some experience modest symptoms, such as mild anemia or a few small bone lesions. Many, but not all, patients with SMM progress to multiple myeloma. The risk of progression is about 10% each year for the first 5 years following diagnosis, 3% between years 5 and 10 and about 1% in subsequent years.

### Combination Therapy

In some embodiments, belumosudil is administered in combination with antimyeloma agents such as anti-CD38 antibodies or IMiDs.

In some embodiments, belumosudil is administered in combination with a an anti-CD38 antibody (e.g., a monoclonal antibody). Examples of anti-CD38 antibodies include, but are not limited to, isatuximab, daratumumab, MOR202, and mezagitamab (TAK-079).

“Isatuximab”, a CD38-directed cytolytic antibody, is a chimeric resurfaced/humanized immunoglobulin GI (IgG1) monoclonal antibody (mAb). Isatuximab can be produced from a mammalian cell line (Chinese hamster ovary, CHO) using a fed-batch production process. Isatuximab is composed of two identical immunoglobulin kappa light chains and two identical immunoglobulin gamma heavy chains and has an overall molecular weight of approximately 148 kDa.

SARCLISA® (isatuximab) injection is a sterile, preservative-free, clear to slightly opalescent, colorless to slightly yellow solution, essentially free of visible particles in a single-dose vial for intravenous use. Each vial contains either 100 mg/5 mL or 500 mg/25 mL of isatuximab at a concentration of 20 mg/mL with a pH of 6.0. Each mL of solution contains 20 mg isatuximab, histidine (1.46 mg), histidine hydrochloride monohydrate (2.22 mg), polysorbate 80 (0.2 mg), sucrose (100 mg), and water for injection.

Isatuximab binds to CD38 expressed on the surface of hematopoietic and tumor cells, including multiple myeloma cells. Isatuximab induces apoptosis of tumor cells and activation of immune effector mechanisms including antibody-dependent cell-mediated cytotoxicity (ADCC), antibody-dependent cellular phagocytosis (ADCP), and complement dependent cytotoxicity (CDC). Isatuximab inhibits the ADP-ribosyl cyclase activity of CD38. Isatuximab can activate natural killer (NK) cells in the absence of CD38-positive target tumor cells and suppresses CD38-positive T-regulatory cells. The combination of isatuximab and pomalidomide enhanced ADCC activity and direct tumor cell killing compared to that of isatuximab alone in vitro, and enhanced antitumor activity compared to the activity of isatuximab or pomalidomide alone in a human multiple myeloma xenograft model.

In some embodiments, the anti-CD38 antibody is isatuximab and can be administered to the individual intravenously at a dose of 10 mg/kg or 20 mg/kg. In other embodiments, isatuximab can be administered subcutaneously at a dose of 1000 mg or 1400 mg.

The anti-CD38 antibody can be administered in cycles. In some embodiments, the anti-CD38 antibody is isatuximab (in either intravenous or subcutaneous form) and is administered in cycles of 28 days. In the first cycle, isatuximab is administered once per week (QW) for example on days 1, 8, 15, and 22 of the cycle. In subsequent cycles, isatuximab can be administered twice per week (Q2W), e.g., on days 1 and 15 of the cycle. After 12 months of isatuximab administration, isatuximab administration can be reduced to once every 4 weeks (Q4W), e.g., on day of and administering the isatuximab at a dose of 10 mg/kg on Day 1 (e.g., Q4W), while maintaining safety and efficacy.

In some embodiments, the anti-CD38 antibody is daratumumab and can be administered to the individual intravenously at a dose of 16 mg/kg. In other embodiments, daratumumab can be administered subcutaneously at a dose of 1800 mg. Daratumumab can be administered in a dosing regimen according to the published prescribing information (published at www.accessdata.fda.gov) for daratumumab injection for intravenous use or daratumumab and hyaluronidase-fihj for the intravenous and subcutaneous formulations, respectively.

In some embodiments, belumosudil is administered in combination with an immunomodulatory drug (sometimes referred to herein as IMiD). Examples of immunomodulatory drugs include, but are not limited to, pomalidomide, lenalidomide, and thalidomide.

“Pomalidomide” is a thalidomide analog. The chemical name is (RS)-4-Amino-2-(2,6-dioxo-piperidin-3-yl)-isoindoline-1,3-dione and it has the following chemical structure:

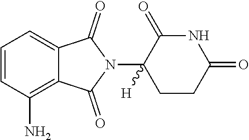 [SMILES: Nc1cccc2c1C(=O)N(C1CCC(=O)NC1=O)C2=O]

The empirical formula for pomalidomide is C₁₃H₁₁N₃O₄ and the gram molecular weight is 273.24. Pomalidomide is a yellow solid powder. It has limited to low solubility into organic solvents and it has low solubility in all pH solutions (about 0.01 mg/mL). Pomalidomide has a chiral carbon atom which exists as a racemic mixture of the R(+) and S(−) enantiomers. Pomalidomide is available in 1-mg, 2-mg, 3-mg, and 4-mg capsules for oral administration. Each capsule contains pomalidomide as the active ingredient and the following inactive ingredients: mannitol, pregelatinized starch, and sodium stearyl fumarate. The 1-mg capsule shell contains gelatin, titanium dioxide, FD&C blue 2, yellow iron oxide, white ink, and black ink. The 2-mg capsule shell contains gelatin, titanium dioxide, FD&C blue 2, yellow iron oxide, FD&C red 3, and white ink. The 3-mg capsule shell contains gelatin, titanium dioxide, FD&C blue 2, yellow iron oxide, and white ink. The 4-mg capsule shell contains gelatin, titanium dioxide, FD&C blue 1, FD&C blue 2, and white ink.

“Dexamethasone” is an adrenocortical steroid. The chemical name is chemically as 9-fluoro-11β,17,21-trihydroxy-16α-methylpregna-1,4-diene-3,20-dione and it has the following chemical structure:

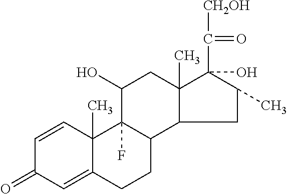

The empirical formula for dexamethasone is C₂₂H₂₉FO₅ and the gram molecular weight is 392.47. Dexamethasone is a white to practically white, odorless, crystalline powder that is stable in air and practically insoluble in water. Dexamethasone is available also as a sodium phosphate ester for injection for intravenous, intramuscular, intra-articular, soft-tissue or intralesional use.

### EXAMPLES

### Example 1: Effect of Belumosudil or Pomalidomide on the Proliferation of Multiple Myeloma (MM) Cell Lines

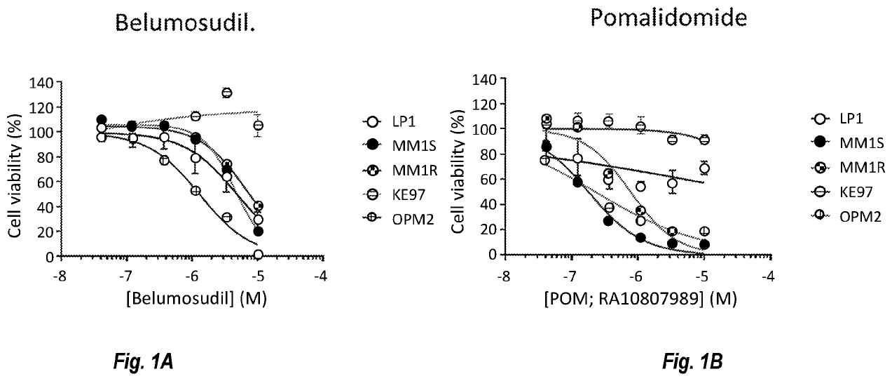

The effect of belumosudil on the proliferation of MM was assessed in vitro on a panel of MM cell lines with different genetic backgrounds (LP1, MM1R, MM1S, OPM2). Cell viability was measured by CellTiter-Glo, Luminescent Cell Viability Assay normalized with DMSO. Cells were treated for 8 days with either belumosudil or pomalidomide. Treatment for 8 days with belumosudil led to MM cell line growth inhibition, in dose dependent manner with an IC₅₀ varying from 1 to 7 mM, as shown in FIG. 1A. As shown in FIG. 1B, pomalidomide also showed growth inhibition in all of these MM cell lines except LP1, which is a known immunomodulatory drug (IMiD) resistant cell line (Leukemia (2014) 28, 1129-1174)), and KE97. Belumosudil showed growth inhibition on LP1, which suggests that there is no cross resistance of belumosudil and IMiDs.

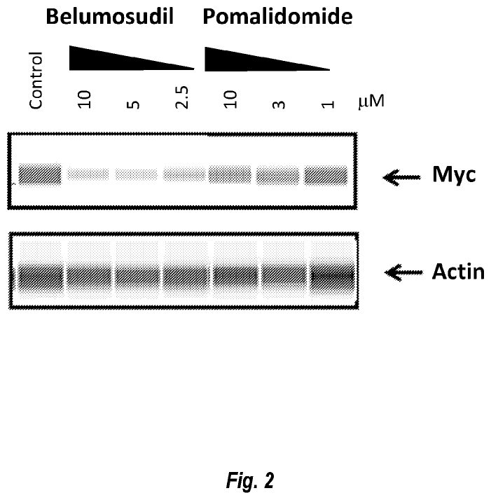

Consistent with MM growth inhibition, in LP1 cells belumosudil induced a dramatic decrease of C-myc protein, a major driver of MM tumors. In contrast, pomalidomide showed a very moderate effect on C-myc protein in LP1 cells. These results are shown in FIG. 2.

Overall, these data demonstrate that belumosudil induces growth inhibition effect on large panel of MM cell lines. Belumosudil has no cross resistance with IMiDs, suggesting that belumosudil could be used for treatment of MM either post-IMiD treatment or in combination with IMiDs.

### Example 2: CD38 Expression on MM Cells and Proliferation/Viability of MM Cell Lines and NK Cells

In vitro assays were performed to assess: 1) the effect of belumosudil (alone or in combination with isatuximab) on the expression of CD38 on MOLP-8 MM cells; 2) the effect of belumosudil (alone or in combination with isatuximab) on the proliferation/viability of MOLP-8 cells; 3) the effect of belumosudil on the proliferation/viability of different MM cell lines (LP1 and RPMI8226); 4) the effect of belumosudil on the proliferation/viability of healthy donors-derived NK cells (HD NK cells).

### Methods

### MM Cell Lines:

MOLP-8 (DSMZ, #ACC569) were maintained in RPMI1640 complete medium (RPMI1640 supplemented with 20% FBS—Eurobio #CVFSVF06-01 and 1% L-glutamine—Gibco #25030-081), incubated at 37° C. with 5% CO2 before being used.

LP1 (DSMZ, #ACC41) were maintained in IMDM medium supplemented with 20% FBS (Eurobio #CVFSVF06-01) and 1% L-glutamine (Gibco #25030-081), incubated at 37° C. with 5% CO₂ before being used.

RPM18226 (ATCC, #CCL155) were maintained in RPMI1640 complete medium, incubated at 37° C. with 5% CO₂ before being used.

Before treatment, MM cells were centrifuged for 5 minutes at 300 g, counted, and resuspended in RPMI1640 complete medium to have 50000 cells/100 μl.

### Human HD NK Cell Isolation:

Healthy donor buffy coats were supplied by EFS Ile de France. Immediately after reception, the qualified buffy coats were put under gentle agitation overnight at room temperature. The following day, peripheral blood mononuclear cells (PBMCs) were isolated by Ficoll (Cytiva #17-1440-02) density gradient centrifugation. Human NK cells were purified from PBMCs with the MACSXpress Whole Blood NK cell isolation kit from Miltenyi Biotec (#130-127-695) by negative selection using manual magnetic labelling and the MACSxpress separator (Miltenyi Biotec #130-098-308). Then, purified NK cells were cultured in RPMI1640 medium supplemented with 10% fetal bovine serum (FBS) (Eurobio #CVFSVF06-01) and 1% of L-Glutamine (Gibco #25030-081). The cells were left to rest overnight at 37° C. with 5% CO₂ before being used.

### Cell Treatment:

Compounds and cells were then added to the appropriate wells of a Corning™ 96-Well Clear Ultra Low Attachment Microplate (Corning, #7007) (final volume of each well: 200 μl).

To assess the effect of belumosudil and/or isatuximab on the expression of CD38 on MOLP-8, as well as on the proliferation/viability of MOLP-8, compounds and cells were added to the appropriate wells in the following order: (1) isatuximab (or isotype control) (previously diluted in RPMI1640 complete medium) was added to the appropriate wells to have a final concentration of 20 μg/ml; (2) belumosudil (or vehicle control—DMSO) was added to the appropriate wells to have final concentrations of 1.1, 3.3, 10, 20 or 25 μM; and (3) MOLP-8 (50000 cells/well) were then added to the appropriate wells.

To assess the effect of belumosudil on the proliferation/viability of LP1, RPM18226, as well as HD NK cells, compounds and cells were added to the appropriate wells in the following order: (1) belumosudil (or vehicle control—DMSO) was added to the appropriate wells to have final concentrations of 10, 15, 20, 25 and 40 μM; (2) LP1 or RPM18226 or HD NK cells (50000 cells/well) were then added to the appropriate wells; and (3) the plate was then centrifuged for 1 minute at 100 g before being placed into an incubator at 37° C. with 5% CO₂ for up to 4 days.

### Flow Cytometry:

After treatment, cells were collected and added to U bottom plates for antibody labelling.

Then, cells were centrifuged for 5 minutes at 300 g, the supernatant removed and 200 μl of AutoMACS Running buffer (Miltenyi Biotec, #130-091-221) added to each well. The process was repeated twice. The cells were then centrifuged for 5 minutes at 300 g, the supernatant removed and 100 μl of mouse anti-human CD38 primary antibody were added to the wells for 30 min at 4° C. Then, 100 μl of AutoMACS Running buffer were added, the cells were centrifuged for 5 minutes at 300 g, the supernatant removed and 200 μl of AutoMACS Running buffer added to each well. The cells were then centrifuged for 5 minutes at 300 g, the supernatant removed and 100 μl of AF488 conjugated AffiniPure Goat anti-mouse IgG secondary antibody (Jackson Immuno Research #115-545-164) was added to the wells for 20 min at 4° C. Then, 100 μl of AutoMACS Running buffer were added, the cells were centrifuged for 5 minutes at 300 g, the supernatant removed and 200 μl of AutoMACS Running buffer added to each well. At the end, cells were centrifuged again for 5 minutes at 300 g and resuspended in 80 μl of DPBS.

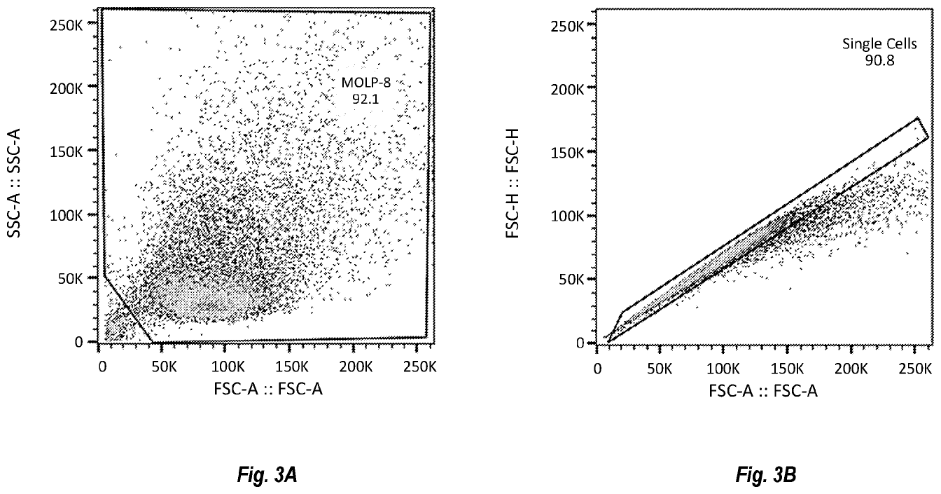

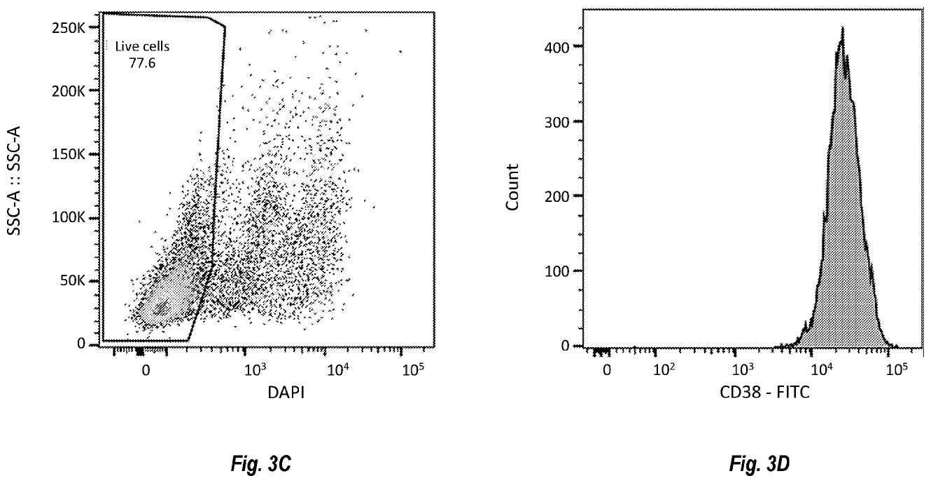

To additionally assess cell viability, DAPI (Miltenyi Biotec, #130-111-570) was added in each well before plate reading by flow cytometry. Samples were then analyzed with the MACSQuant 16 flow cytometer (Miltenyi Biotec). Data were analyzed with FlowJo software using the following strategy shown in FIGS. 3A-3D: (A) MOLP-8 cells excluding debris were gated, followed by gating for single cells (B), from single cells, viable DAPI^{neg} cells were quantified (C), and from DAPI^{neg} cells, the expression of CD38 was quantified with FITC fluorochrome (D) and expressed as median of fluorescence intensity (MFI).

### Results

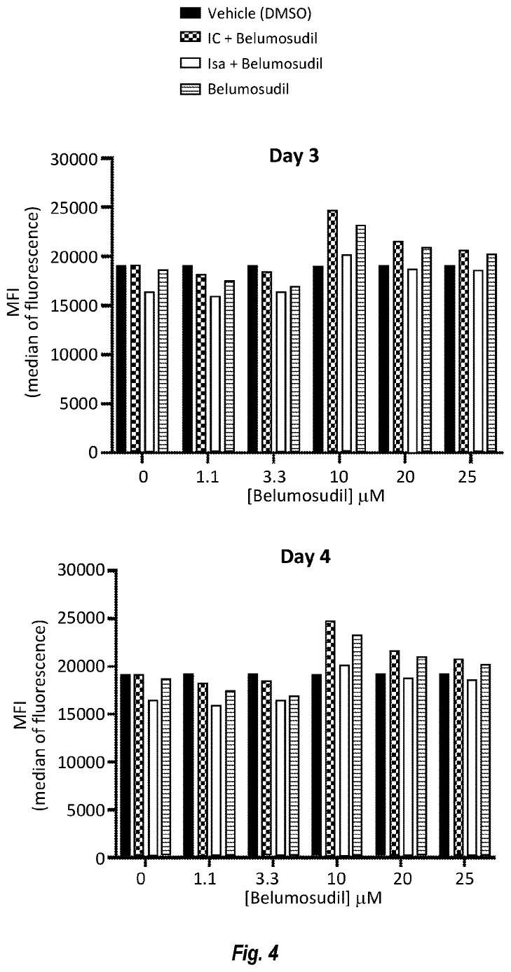

As shown in FIG. 4, CD38 expression decreases on MOLP-8 cells after 3 or 4 days of treatment with isatuximab (20 μg/ml) alone compared to controls. Belumosudil at 1.1 and 3.3 μM in combination with isatuximab (20 μg/ml) does not affect CD38 expression on MOLP-8 cells after 3 and 4 days of treatment. Belumosudil induces an increase of CD38 expression on the surface of MOLP-8 cells at the concentration of 10 μM after 3 and 4 days of treatment. Belumosudil at 20 μM and 25 μM also induces increase of CD38 expression on MOLP-8 but to a lesser extent as compared to 10 μM. Importantly, belumosudil (at 10, 20 and 25 μM) in combination with isatuximab (20 μg/ml) preserves CD38 expression on the surface of MOLP-8 cells after 3 and 4 days of treatment.

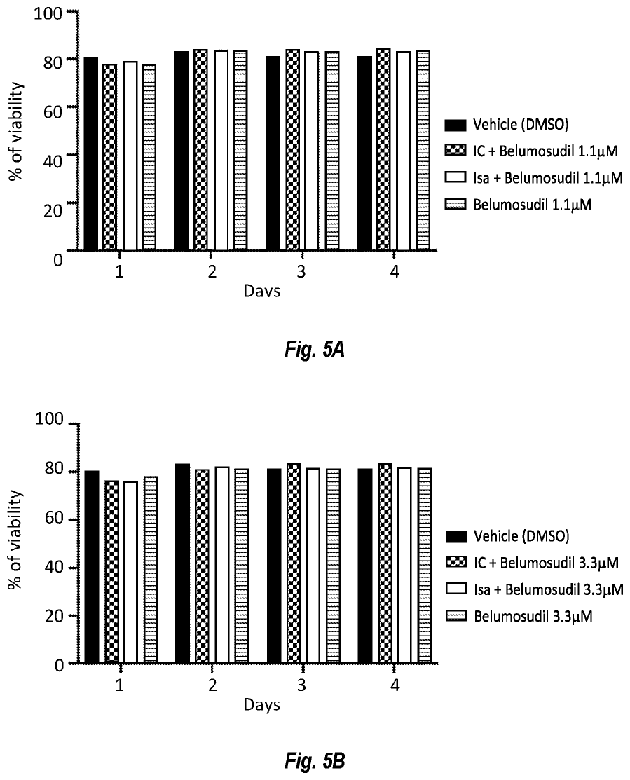

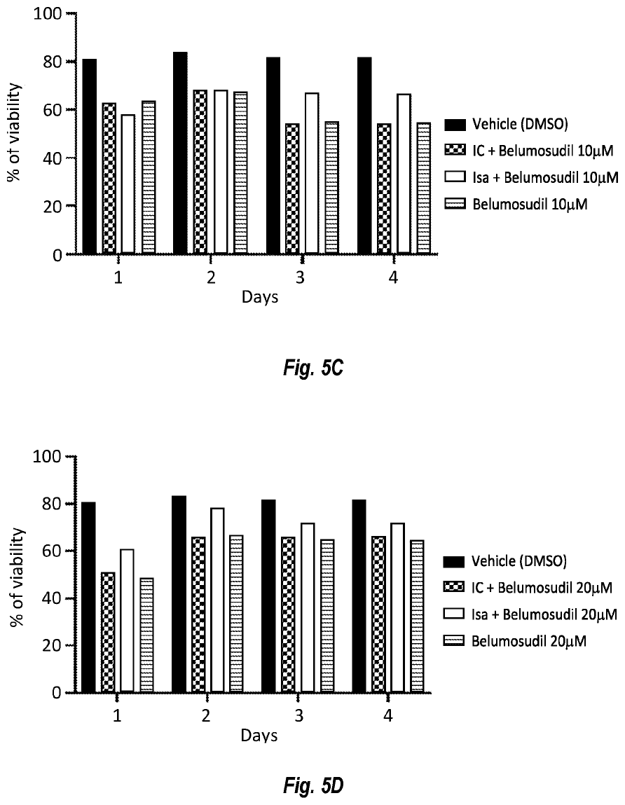

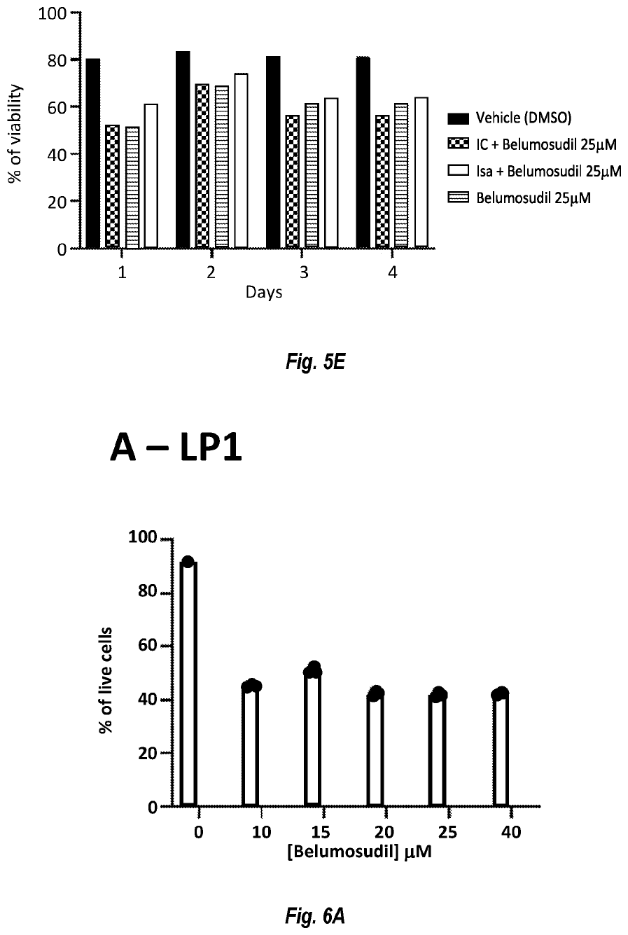

As shown in FIGS. 5A-5E, no impact of belumosudil treatment was observed on MOLP-8 cells viability at low concentrations (1.1 and 3.3 μM) (FIGS. 5A and 5B, respectively). Limited impact of belumosudil on MOLP-8 cells viability (reduction) was observed at higher concentrations (10, 20 and 25 μM) starting at 1 day after treatment (FIGS. 5C, 5D and 5E, respectively).

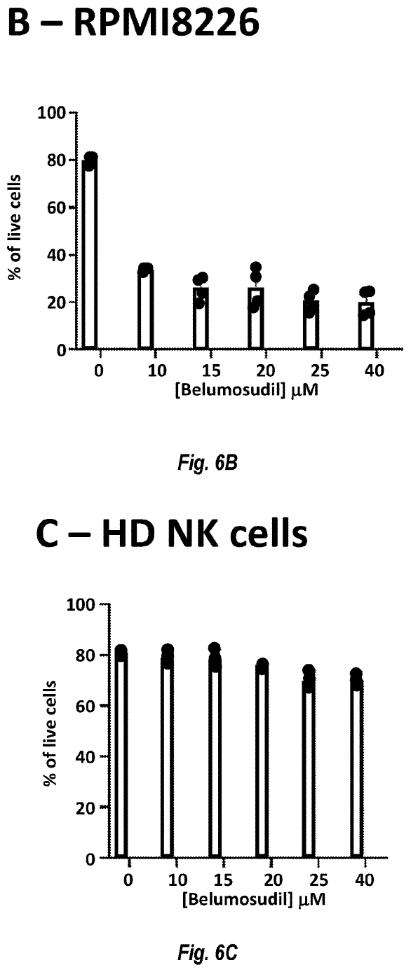

As shown in FIGS. 6A-6C, belumosudil strongly reduces viability of LP1 and RPMI8226 cells, which are known immunomodulatory drug (IMiD) resistant cell lines (Leukemia (2014) 28, 1129-1174; Blood. 2011; 117(19): 5157-5165), starting at 10 μM after 4 days of treatment (FIGS. 6A and 6B). Belumosudil does not impact or has a very limited impact on HD NK cells viability (max 10% reduction at high concentrations 25-40 μM) after 4 days of treatment.

### Example 3: Apoptosis of MOLP-8 MM Cells

The pro-apoptotic effect of belumosudil (alone or in combination with isatuximab) on MOLP-8 MM cells was assessed in vitro.

### Methods

### MM Cell Lines:

MOLP-8 (DSMZ, #ACC569) were maintained in RPMI1640 complete medium (RPMI1640 supplemented with 20% FBS—Eurobio #CVFSVF06-01 and 1% L-glutamine—Gibco #25030-081), incubated at 37° C. with 5% CO₂ before being used.

Before treatment, MM cells were centrifuged for 5 minutes at 300 g, counted, and resuspended in RPMI1640 complete medium to have 50000 cells/100 μl.

### Cell Treatment:

Compounds and cells were then added to the appropriate wells of a Corning™ 96-Well Clear Ultra Low Attachment Microplate (Corning, #7007) (final volume of each well: 200 μl) in the following order: (1) isatuximab (or isotype control) (previously diluted in RPMI1640 complete medium) was added to the appropriate wells to have a final concentration of 20 μg/ml; (2) belumosudil (or vehicle control—DMSO) was added to the appropriate wells to have final concentrations of 1.1, 3.3, 10, 20 or 25 μM; (3) MOLP-8 (50000 cells/well) were then added to the appropriate wells; and (4) the plate was then centrifuged for 1 minute at 100 g before being placed into an incubator at 37° C. with 5% CO₂ for up to 4 days.

### Flow Cytometry:

Apoptosis was assessed using the AnnexinV Apoptosis detection kit eFluor450 (Invitrogen, #88-8006-74).

After treatment, cells were collected and added to U bottom plates for antibody labelling.

Then, cells were centrifuged for 5 minutes at 300 g, the supernatant removed and 200 μl of D-PBS added to each well. The cells were then centrifuged for 5 minutes at 300 g, the supernatant removed and 200 μl of 1× buffer (from the AnnexinV Apoptosis detection kit) added to each well. The cells were then centrifuged for 5 minutes at 300 g, the supernatant removed and 100 μl of AnnexinV labelling solution (from the AnnexinV Apoptosis detection kit) were added to the wells for 20 min at 4° C. (following manufacturer's instructions).

Then, 100 μl of 1× buffer were added, the cells were centrifuged for 5 minutes at 300 g, the supernatant removed and 200 μl of 1× buffer added to each well. The cells were then centrifuged for 5 minutes at 300 g, the supernatant removed and resuspended in 80 μl of D-PBS.

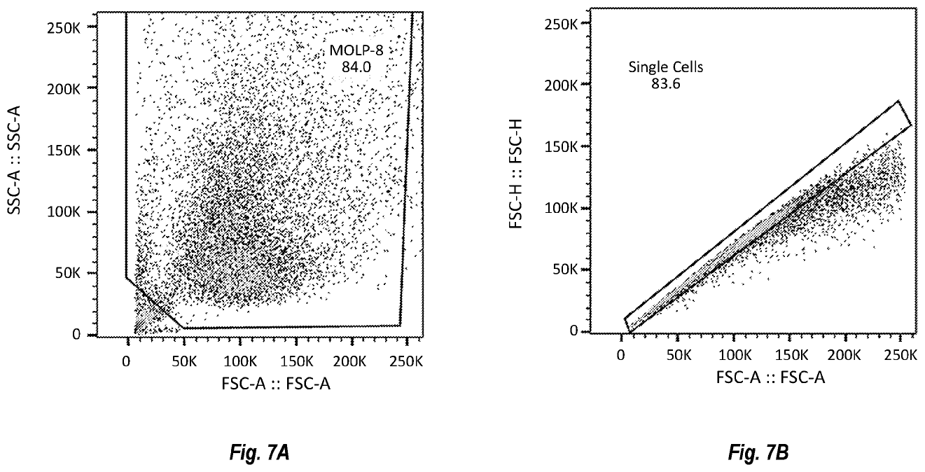

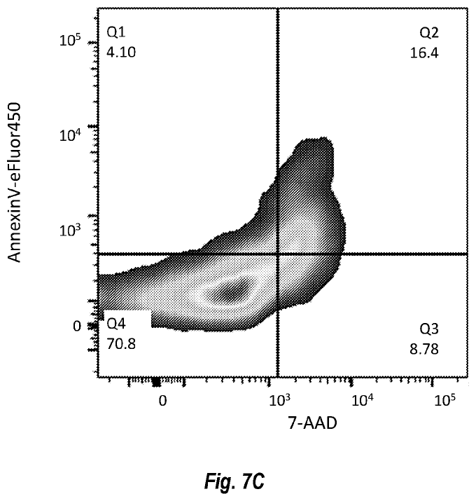

In addition, 7-AAD (from the AnnexinV Apoptosis detection kit) was added in each well before plate reading by flow cytometry. Samples were then analyzed with the MACSQuant 16 flow cytometer (Miltenyi Biotec). Data were analyzed with FlowJo software using the following strategy in FIGS. 7A-7C: MOLP-8 cells excluding debris were gated (A), followed by gating for single cells(B). From single cells, early apoptotic 7-AAD^{neg} and Annexin V-eFluor450^{pos} (C, Q1) and late apoptotic 7-AAD^{POs} and Annexin V-eFluor450^{pos} (C, Q2) cells were quantified (C). The percentage of early and late apoptotic cells is represented in the graphs.

### Results

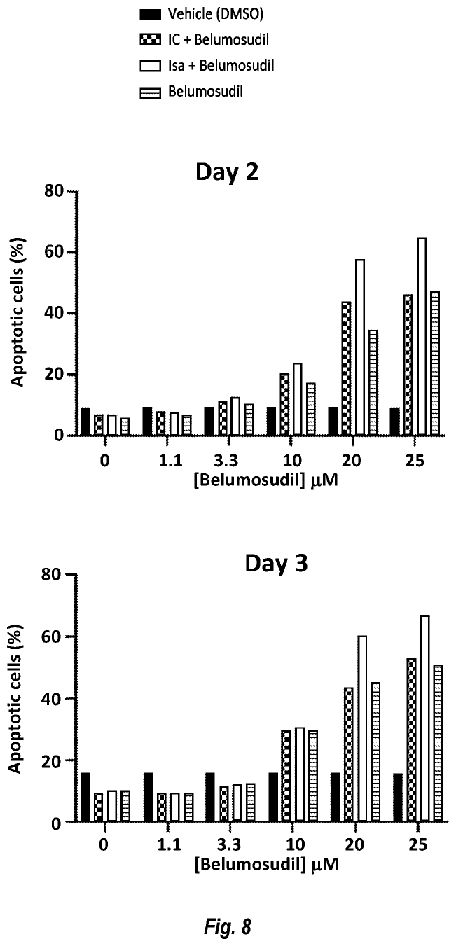

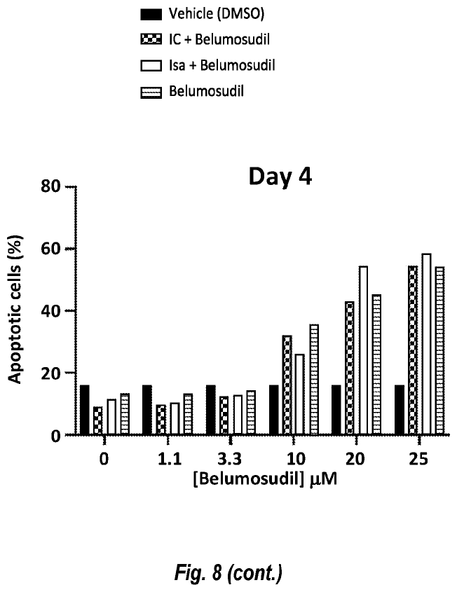

As shown in FIG. 8, belumosudil at 10, 20 and 25 μM induces apoptosis of MOLP-8 after 2, 3, and 4 days of treatment, and in a dose-dependent manner. Belumosudil at 20 and 25 μM in combination with isatuximab (20 μg/ml) further increase apoptosis of MOLP-8 MM cells after 2, 3, and 4 days of treatment, and in a dose-dependent manner.

### Example 4: Long Term Cytotoxicity Against LP-1 MM Cells

Cytotoxicity against LP-1 RFP multiple myeloma cells (target cells) was assessed in presence of healthy donor (HD)-derived NK cells (effector cells) after treatment with isatuximab in combination with belumosudil. Cytotoxicity was measured over time by Incucyte® live-cell imaging and analysis system (Essen Bioscience).

### Methods

### Generation of RFP+LP1 MM Cells:

LP-1 RFP cells were generated by infecting LP-1 cells (DSMZ, #ACC41) with Incucyte® Nuclight Red Lentivirus (Sartorius) to express red fluorescent protein (RFP). LP-1 RFP cells were maintained in IMDM medium (Gibco, #12440053) supplemented with 20% fetal bovine serum (FBS heat inactivated, Biowest, #S181H-100), 1% L-Glutamine (Gibco, #25030-024), and incubated at 37° C. with 5% CO₂. LP-1 RFP cells were centrifuged for 5 minutes at 300 g, resuspended in RPMI1640 complete medium (RPMI1640 supplemented with 10% fetal bovine serum—FBS, Biowest, #S181H-100-, 1% L-Glutamine—Gibco, #25030-024) before being added to Incucyte® plates (details below).

### Human HD NK Cell Isolation:

Healthy donor buffy coats were supplied by EFS Ile de France. Immediately after reception, the qualified buffy coats were put under gentle agitation overnight at room temperature. The following day, peripheral blood mononuclear cells (PBMCs) were isolated by Ficoll (Cytiva #17-1440-02) density gradient centrifugation. Human NK cells were purified from PBMCs with the MACSXpress Whole Blood NK cell isolation kit from Miltenyi Biotec (#130-127-695) by negative selection using manual magnetic labelling and the MACSxpress separator (Miltenyi Biotec #130-098-308). Then, purified NK cells were cultured in RPMI1640 medium supplemented with 10% fetal bovine serum (FBS) (Eurobio #CVFSVF06-01) and 1% of L-Glutamine (Gibco #25030-081). The cells were left to rest overnight at 37° C. with 5% CO₂ before being used.

### Cytotoxicity Assay by Incucyte®:

Cytotoxicity was evaluated using the Incucyte® live-cells analysis system which allows to quantify the number of live fluorescent target cells over time.

Compounds and cells were added to the appropriate wells of an Incucyte® plate (Poly-D lysine treated 96-well flat bottom microplate CellCoat™, Greiner Bio-One; #655946) in the following order (final volume of each well: 200 μl): (1) isatuximab (or isotype control) (previously diluted in RPMI1640 complete medium) was added to the appropriate wells to have a final concentration of either 1, 10, 100 or 1000 ng/ml; (2) belumosudil (previously diluted in RPMI1640 complete medium) was then added to the appropriate wells to have a final concentration of either 1.1, 3.3 and 10 μM; (3) LP-1 RFP cells (target cells, T) were then added to each well (to have 20000 LP1-RFP cells/well); (4) HD NK cells (effector cells, E) were then added to the appropriate wells (60000 cells were added to have an E:T ratios of 3:1), HD NK cells were not added in some wells to test the direct effect of belumosudil against LP-1 RFP cells.

The Incucyte® plate was then centrifuged for 1 minute at 100 g before being placed into an Incucyte® (IncucyteS3, EssenBio) which was housed within a dedicated incubator at 37° C. with 5% CO₂. Images (4 images/well) were taken every 4 hours with a 10× objective and a standard scan type using the phase and red channels. The growth of LP-1 RFP target cells was monitored by fluorescent imaging up to 90 hours, and the number of live target cells quantified using the IncucyteS3 software and normalized to the number of live target cells time zero.

### Results

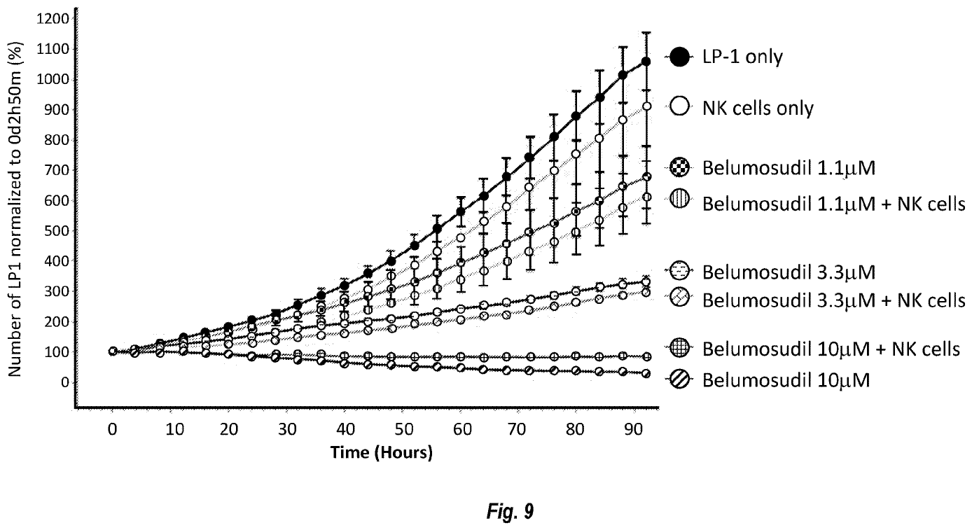

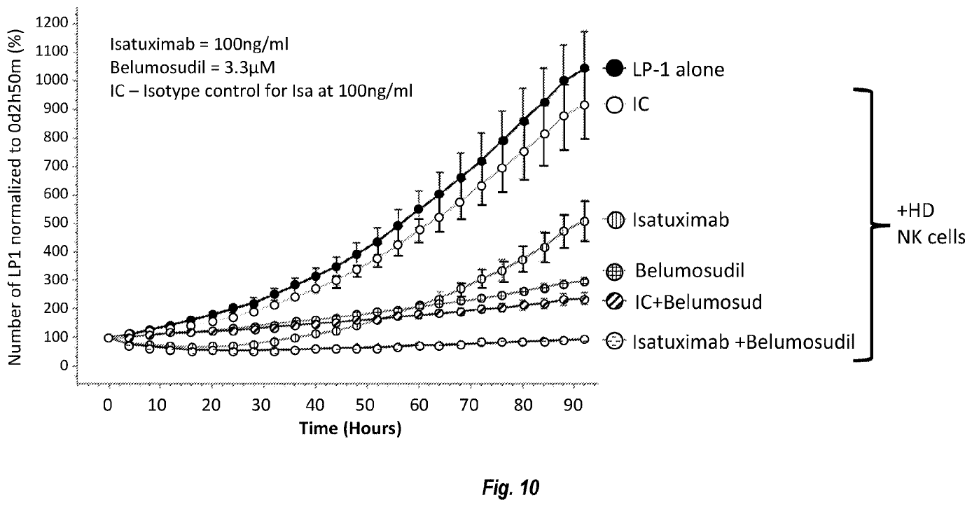

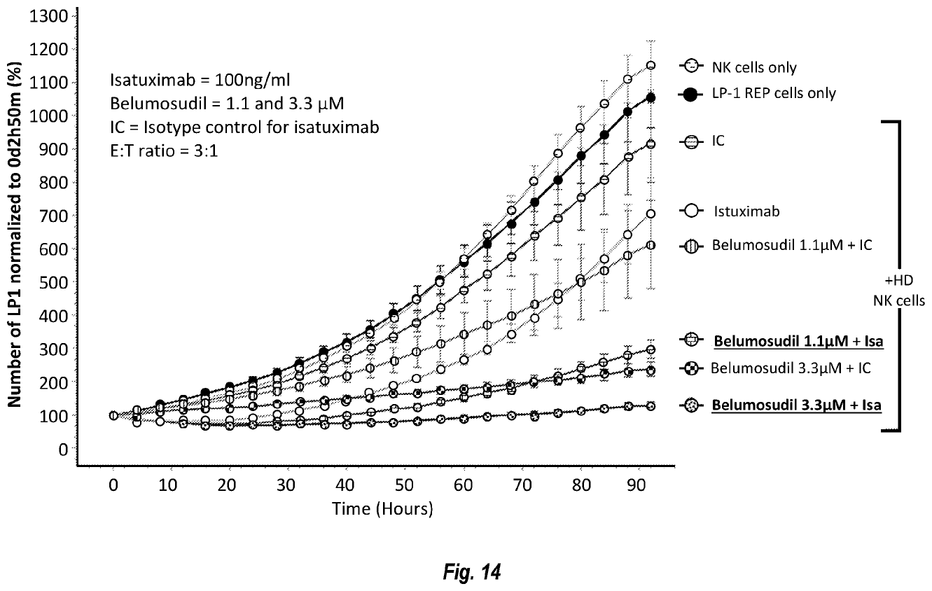

As shown in FIGS. 9 and 10, belumosudil induces killing of LP-1 in a dose dependent manner over time independently on the presence of HD NK cells (FIG. 9). Belumosudil (1.1 or 3.3 μM) in combination with isatuximab (100 ng/ml) further increases LP-1 cell killing over time in presence of HD NK cells (FIG. 10 and FIG. 14).

### Example 5: The Effect of Belumosudil Compared to a Pan-ROCK Inhibitor (Y27632) on Proliferation/Viability of Multiple Myeloma (MM) Cell Lines and NK Cells

The effect of belumosudil compared to a pan-ROCK inhibitor (Y27632) on the proliferation and/or viability of different MM cell lines (MOLP-8, LP-1 and RPMI-8226) was assessed, as was the effect of belumosudil compared to a pan-ROCK inhibitor (Y27632) on the proliferation and/or viability of healthy donor-derived NK cells (HD NK cells).

### Methods

### MM Cell Lines:

MOLP-8 (DSMZ, #ACC569) were maintained in RPMI1640 complete medium (RPMI1640 supplemented with 20% FBS—Eurobio #CVFSVF06-01 and 1% L-glutamine—Gibco #25030-081), incubated at 37° C. with 5% CO₂ before being used.

LP-1 (DSMZ, #ACC41) were maintained in IMDM medium supplemented with 20% FBS (Eurobio #CVFSVF06-01) and 1% L-glutamine (Gibco #25030-081), incubated at 37° C. with 5% CO₂ before being used.

RPMI-8226 (ATCC, #CCL155) were maintained in RPMI1640 complete medium, incubated at 37° C. with 5% CO₂ before being used.

Before treatment, MM cells were centrifuged for 5 minutes at 300 g, counted, and resuspended in RPMI1640 complete medium to have 50000 cells/100 μl.

### Human HD NK Cell Isolation:

Healthy donor buffy coats were supplied by EFS Ile de France. Immediately after reception, the qualified buffy coats were put under gentle agitation overnight at room temperature. The following day, peripheral blood mononuclear cells (PBMCs) were isolated by Ficoll (Cytiva #17-1440-02) density gradient centrifugation. Human NK cells were purified from PBMCs with the MACSXpress Whole Blood NK cell isolation kit from Miltenyi Biotec (#130-127-695) by negative selection using manual magnetic labelling and the MACSxpress separator (Miltenyi Biotec #130-098-308). Then, purified NK cells were cultured in RPMI1640 medium supplemented with 10% foetal bovine serum (FBS) (Eurobio #CVFSVF06-01) and 1% of L-Glutamine (Gibco #25030-081). The cells were left to rest overnight at 37° C. with 5% CO₂ before being used.

### Cell Treatment:

Compounds and cells were then added to the appropriate wells of a Corning™ 96-Well Clear Ultra Low Attachment Microplate (Corning, #7007) (final volume of each well: 200 μl). To assess the effect of Belumosudil or Y27632 on the proliferation and/or viability of MOLP-8, LP-1, RPMI-8226, as well as HD NK cells, compounds and cells were added to the appropriate wells in the following order: Belumosudil or Y27632 (Merck, #688000) or vehicle control (DMSO) was added to the appropriate wells to have final concentrations of 5 (only for LP-1 cells) 10, 15, 20, 25 and 40 μM. MOLP-8 or LP-1 or RPMI-8226 or HD NK cells (50000 cells/well) were then added to the appropriate wells. The plate was then centrifuged for 1 minute at 100 g before being placed into an incubator at 37° C. with 5% CO₂ for up to 4 days.

### Flow Cytometry:

After treatment, cells were collected and added to U bottom plates for antibody labelling.

Then, cells were centrifuged for 5 minutes at 300 g, the supernatant removed and 200 μl of AutoMACS Running buffer (Miltenyi Biotec, #130-091-221) added to each well. The process was repeated twice. The cells were then centrifuged for 5 minutes at 300 g, the supernatant removed and 100 μl of mouse anti-human CD38 primary antibody (internal Sanofi production) were added to the wells for 30 min at 4° C. (only for belumosudil treated cells). Then, 100 μl of AutoMACS Running buffer were added, the cells were centrifuged for 5 minutes at 300 g, the supernatant removed and 200 μl of AutoMACS Running buffer added to each well. The cells were then centrifuged for 5 minutes at 300 g, the supernatant removed and 100 μl of AF488 conjugated AffiniPure Goat anti-mouse IgG secondary antibody (Jackson Immuno Research #115-545-164) was added to the wells for 20 min at 4° C. Then, 100 μl of AutoMACS Running buffer were added, the cells were centrifuged for 5 minutes at 300 g, the supernatant removed and 200 μl of AutoMACS Running buffer added to each well. At the end, cells were centrifuged again for 5 minutes at 300 g and resuspended in 80 μl of DPBS.

To additionally assess cell viability, DAPI (Miltenyi Biotec, #130-111-570) was added in each well before plate reading by flow cytometry. Samples were then analyzed with the MACSQuant 16 flow cytometer (Miltenyi Biotec).

Data were analyzed with FlowJo software as shown in FIG. 3: (FIG. 3A) MOLP-8 cells excluding debris was gated, followed by gating for single cells (FIG. 3B). From the single cells, viable DAPI^{neg} cells were quantified (FIG. 3C). From DAPI^{neg} cells, the expression of CD38 was quantified (only for belumosudil treated cells) with FITC fluorochrome (FIG. 3D) and expressed as median of fluorescence intensity (MFI).

### Results

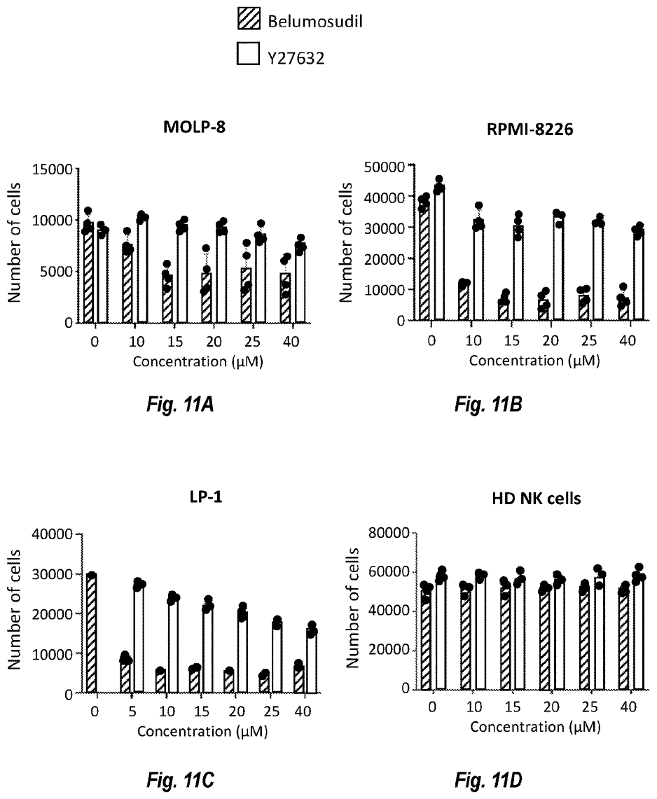

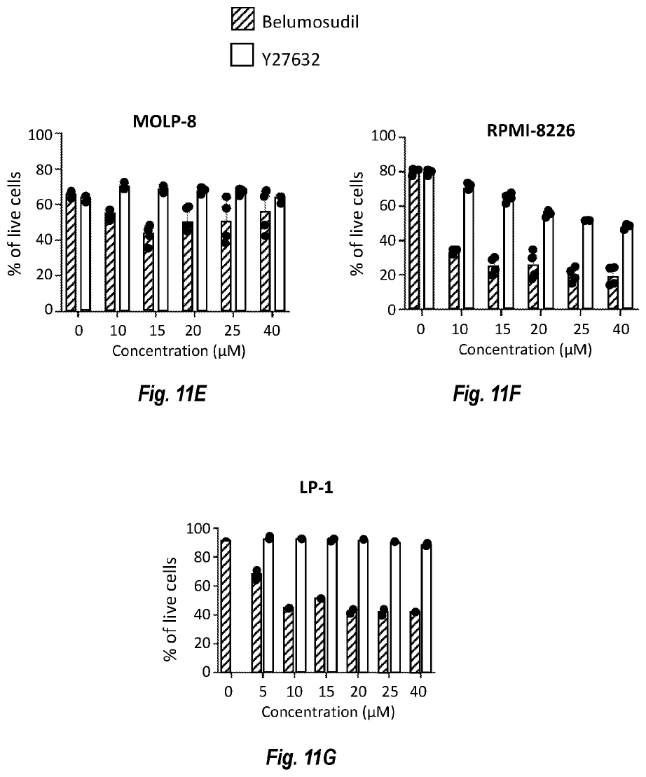

The data in FIGS. 11A-11G show that belumosudil reduces viability (both number of cells and % of live cells) of MOLP-8, RPMI-8226 and LP-1 cells starting at 10 μM (5 μM for LP-1) after 4 days of treatment; Y27632 does not impact viability (either number of cells or % of live cells) of MOLP-8, RPMI-8226 after 4 days of treatment; and neither belumosudil nor Y27632 impact HD NK cell viability after 4 days of treatment.

### Example 6: Long-Term Cytotoxicity Against RPMI-8226 and MM.1R MM Cell Lines

Cytotoxicity against RPMI-8226 RFP and MM.1R RFP multiple myeloma cells (target cells) was assessed in presence of healthy donor (HD)-derived NK cells (effector cells) after treatment with isatuximab in combination with belumosudil. Cytotoxicity was measured over time by Incucyte® live-cell imaging and analysis system (Essen Bioscience).

### Generation of RFP Positive RPMI-8226 and MM.1R MM Cells:

RPMI-8226 RFP cells were generated by infecting RPMI-8226 cells (ATCC, #CCL 155) with 3rd generation lentivirus HIV-based, VSV-G pseudotyped lentiviral, coding for RFP (mKate2 Lentivirus Reagent—Sartorius ref #4625) to express red fluorescent protein (RFP). MM.1R RFP cells were generated by infecting MM.1R cells (ATCC, #CRL 2975) with 3rd generation lentivirus HIV-based, VSV-G pseudotyped lentiviral, coding for RFP (mKate2 Lentivirus Reagent—Sartorius ref #4625) to express red fluorescent protein (RFP).

RPMI-8226 RFP cells or MM.1R RFP cells were maintained in IMDM medium (Gibco, #12440053) supplemented with 20% foetal bovine serum (FBS heat inactivated, Biowest, #S181H-100), 1% L-Glutamine (Gibco, #25030-024), and incubated at 37° C. with 5% CO₂. RPMI-8226 RFP cells were centrifuged for 5 minutes at 300 g, resuspended in RPMI1640 complete medium (RPMI1640 supplemented with 10% foetal bovine serum—FBS, Biowest, #S181H-100-, 1% L-Glutamine—Gibco, #25030-024) before being added to Incucyte® plates (detailed below).

### Human HD NK Cell Isolation:

Healthy donor buffy coats were supplied by EFS Ile de France. Immediately after reception, the qualified buffy coats were put under gentle agitation overnight at room temperature. The following day, peripheral blood mononuclear cells (PBMCs) were isolated by Ficoll (Cytiva #17-1440-02) density gradient centrifugation. Human NK cells were purified from PBMCs with the MACSXpress Whole Blood NK cell isolation kit from Miltenyi Biotec (#130-127-695) by negative selection using manual magnetic labelling and the MACSxpress separator (Miltenyi Biotec #130-098-308). Then, purified NK cells were cultured in RPMI1640 medium supplemented with 10% foetal bovine serum (FBS) (Eurobio #CVFSVF06-01) and 1% of L-Glutamine (Gibco #25030-081). The cells were left to rest overnight at 37° C. with 5% CO₂ before being used.

### Cytotoxicity Assay by Incucyte®:

Cytotoxicity was evaluated using the Incucyte® live-cells analysis system which allows to quantify the number of live fluorescent target cells over time. Compounds and cells were added to the appropriate wells of an Incucyte® plate (Poly-D lysine treated 96-well flat bottom microplate CellCoat™, Greiner Bio-One; #655946) in the following order (final volume of each well: 200 μl): Isatuximab (or isotype control) (previously diluted in RPMI1640 complete medium) was added to the appropriate wells to have a final concentration of either 10, 100 or 1000 ng/ml; Belumosudil (previously diluted in RPMI1640 complete medium) was then added to the appropriate wells to have a final concentration of either 1.1 and 3.3 M; RFP positive MM tumor cells (target cells, T) were then added to each well (to have 20000 RFP positive cells/well); HD NK cells (effector cells, E) were then added to the appropriate wells (60000 RPMI-8226 RFP cells were added to have an E:T ratio of 3:1; 20000 MM.1R RFP cells were added to have an E:T ratio of 1:1); HD NK cells were not added in some wells to test the test the direct effect of belumosudil against RFP positive tumor cells.

The Incucyte® plate was then centrifuged for 1 minute at 100 g before being placed into an Incucyte® (IncucyteS3, EssenBio) which was housed within a dedicated incubator at 37° C. with 5% CO₂. Images (4 images/well) were taken every 4 hours with a 10× objective and a standard scan type using the phase and red channels. The growth of RFP positive target cells was monitored by fluorescent imaging up to 96 hours, and the number of live target cells quantified using the IncucyteS3 software and normalized to the number of live target cells time zero.

### Results

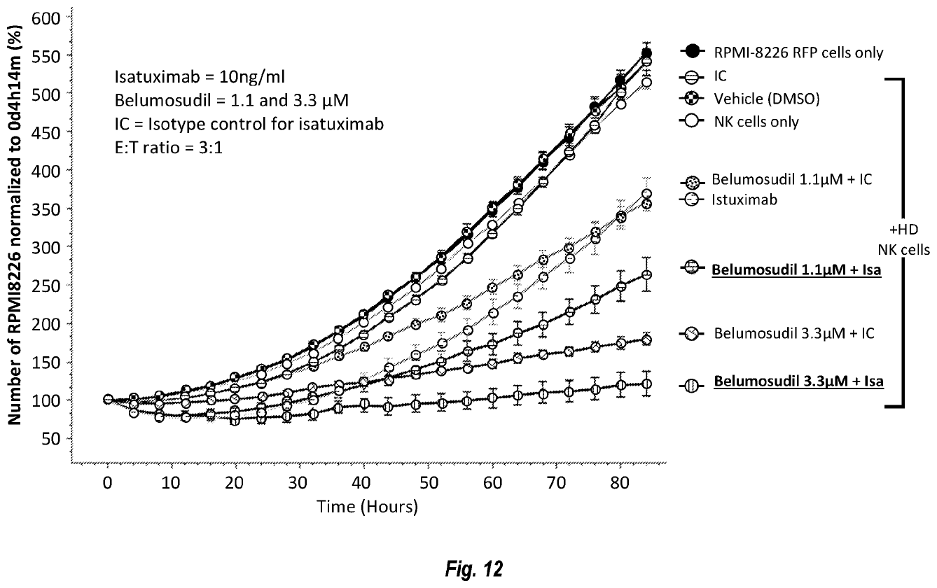

As shown in FIG. 12, belumosudil (1.1 or 3.3p M) induces killing of RPMI-8226 RFP cells over time. Belumosudil (1.1 or 3.3 μM) in combination with isatuximab (10 ng/ml) further increases RPMI-8226 RFP cell killing over time in presence of HD NK cells.

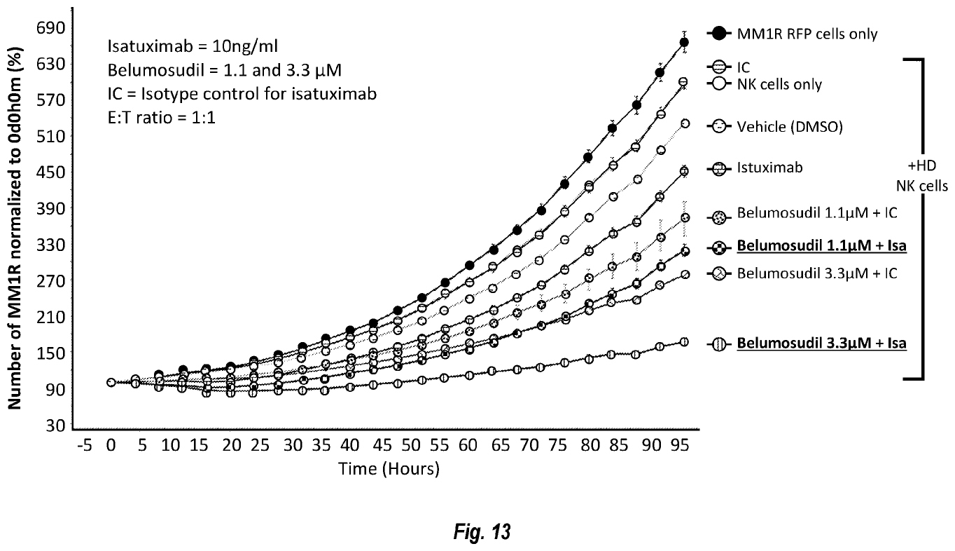

As shown in FIG. 13, belumosudil (1.1 or 3.3 μM) induces killing of MM.1R RFP cells over time. Belumosudil (1.1 or 3.3 μM) in combination with isatuximab (10 ng/ml) further increases MM.1R RFP cell killing over time in presence of HD NK cells.

As shown in FIG. 14, belumosudil (1.1 or 3.3 μM) induces killing of LP-1 RFP cells over time. Belumosudil (1.1 or 3.3 μM) in combination with isatuximab (10 ng/ml) further increases LP-1 RFP cell killing over time in presence of HD NK cells.

### Example 7: Combination of Belumosudil and Dexamethasone in MM Cells

### Methods.

On Day 1, the cells were plated at 3,000 cells per well (50 ul) using 384w plate (Costar, 3765) pre-coated with Poly-D-Lysine, spun down at 300 g for 1 min at room temperature and transferred in Incucyte S3 (37° C.; 5% CO₂; every 2 h kinetic read). Four (4) hours later, the cells were treated using Tecan d300e in dose response with ROCK 2 inhibitor (SAR445761, Belumosudil), or with the Dexamethasone (SelleckChem; S1322) for single agents. For the combination, the dose response of belumosudil in presence of selected concentrations of dexamethasone was used. Cells were further incubated in Incucyte S3 (Sartorius) for an additional 5-6 days at 37° C., 5% CO₂ with the same kinetic readings. At the end, proliferation was measured by added CellTiterGlo (Promega) to assess cell viability or by using Incucyte measuring cell confluency over time. Cell growth was normalized to DMSO (100%) and staurosporine (Sigma; S6942) 1 uM (0%)).

The degree of synergy between belumosudil and dexamethasone was evaluated by synergy finder tool using the excess over the maximum single drug response (HSA) (Lanevski A, et al, Nucleic Acids Research, 488-493, 2020, Vol. 48 (doi: 10.1093/nar/gkaa216).

### Results

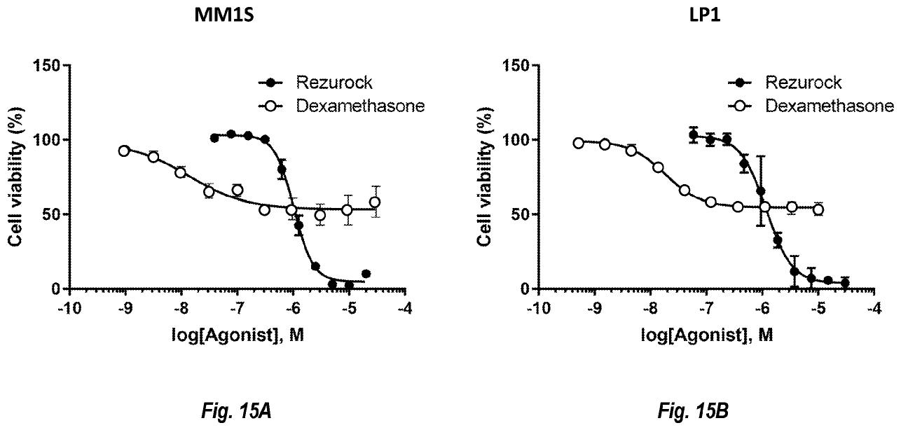

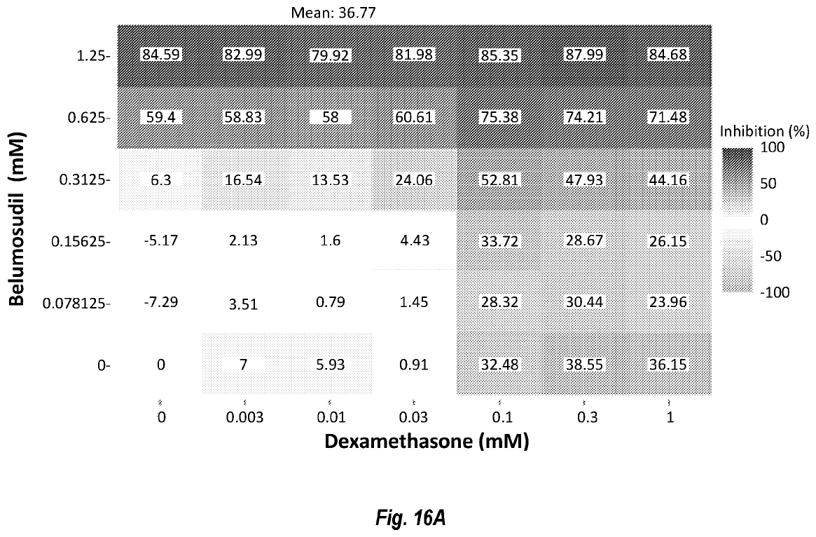

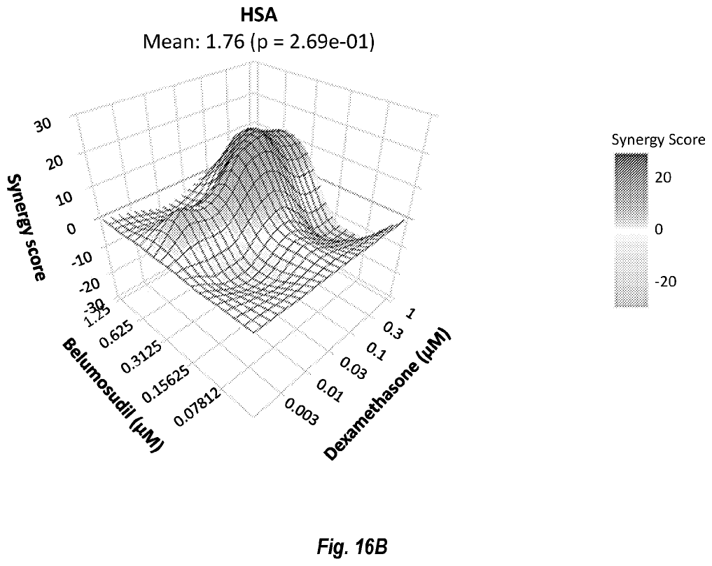

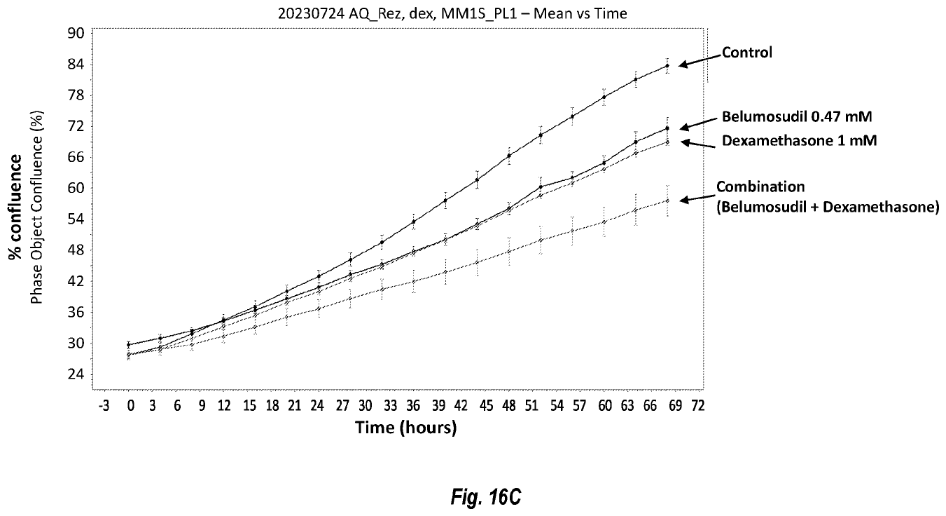

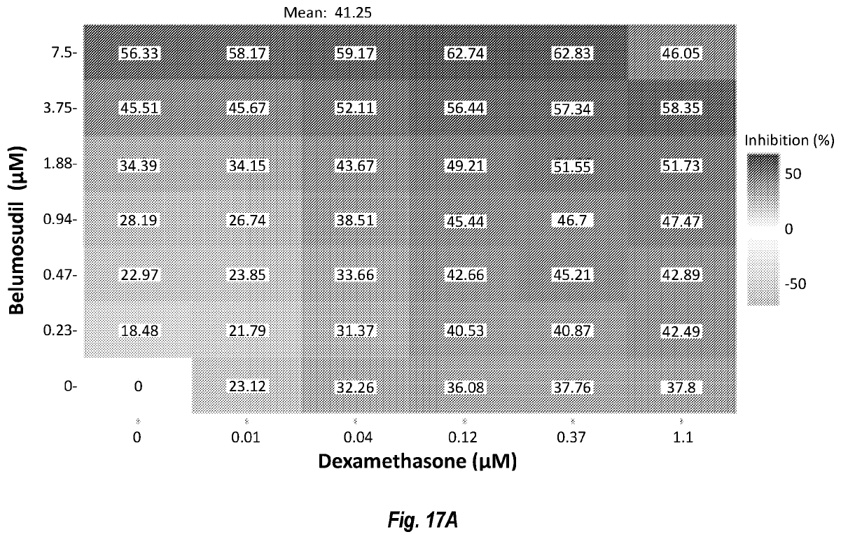

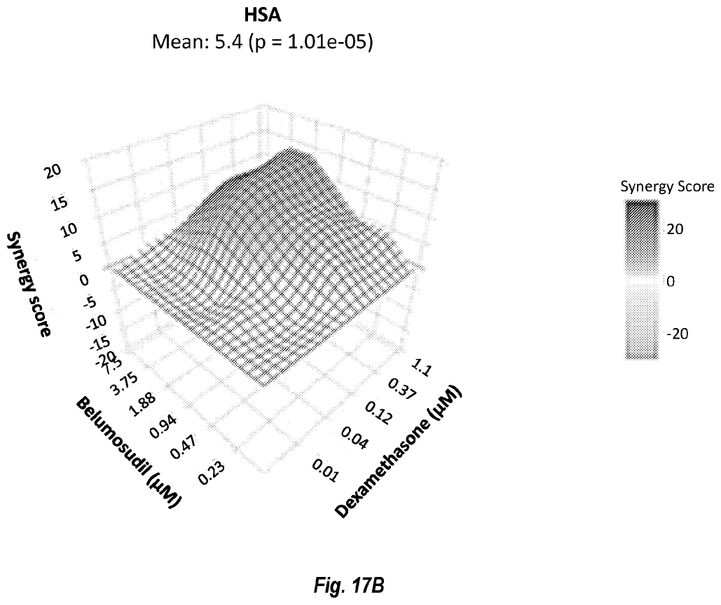

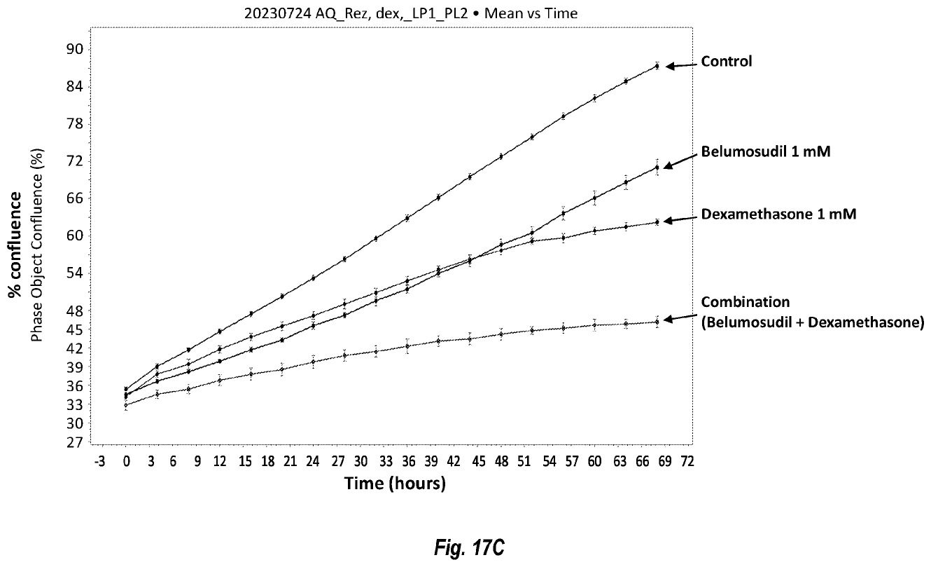

Dexamethasone and belumosudil show single agent growth inhibition activity against multiple myeloma cell lines MM1S and LP1, as shown in FIGS. 15A and 15B, respectively. The combination of belumosudil and dexamethasone shows improved growth inhibition effect in both MM1S and LP1 in a defined range of concentrations, as shown in FIGS. 16A, 16C, 17A, and 17C. Statistical analysis using synergy-finder (HSA) highlighted a positive synergy score of about 1.76 for MM1S and 5.4 for LP-1, as shown in FIGS. 16B and 17B, respectively. Overall these data suggest that the combination of belumosudil and dexamethasone could have a therapeutic benefit for multiple myeloma patients.

Although the present invention has been described in some detail by way of illustration and example for purposes of clarity and understanding, the descriptions and examples should not be construed as limiting the scope of the invention. The disclosures of all patent and scientific literature cited herein are expressly incorporated herein in their entirety by reference.

1. A method of treating multiple myeloma, comprising administering to a subject in need thereof a therapeutically effective amount of 2-{3-[4-(1H-indazol-5-ylamino)-2-quinazolinyl]phenoxy}-N-(propan-2-yl) acetamide, or a pharmaceutically acceptable salt thereof (belumosudil).

2. The method of claim 1, wherein belumosudil is administered as a monotherapy.

3. The method of claim 1, wherein belumosudil is administered in combination with an anti-CD38 antibody.

4. The method of claim 3, wherein the anti-CD38 antibody is daratumumab, isatuximab, MOR202 or mezagitamab (TAK-079).

5. The method of claim 4, wherein the anti-CD38 antibody is isatuximab.

6. The method of claim 5, wherein the isatuximab is administered to the subject intravenously at a dose of 10 mg/kg or 20 mg/kg.

7. The method of claim 5, wherein the isatuximab is administered subcutaneously at a dose of 1000 mg or 1400 mg.

8. The method of any one of claims 1-7, wherein the isatuximab is administered in a 28-day cycle.

9. The method of claim 8, wherein in a first 28-day cycle the isatuximab is administered once per week.

10. The method of claim 9, wherein in subsequent 28-day cycles the isatuximab is administered twice per week.

11. The method of claim 10, wherein after 12 months of isatuximab administration, the isatuximab is administered once every 4 weeks.

12. The method of claim 1, wherein belumosudil is administered in combination with an immunomodulatory drug (IMiD).

13. The method of claim 12, wherein the IMiD is selected from pomalidomide, lenalidomide, thalidomide, iberdomide, and mezigdomide.

14. The method of claim 13, wherein the IMiD is pomalidomide.

15. The method of claim 14, wherein the pomalidomide is administered to the subject at a dose of 4 mg once per day from day 1 to day 21 of each 28-day cycle.

16. The method of claim 14 or 15, wherein the pomalidomide is administered orally.

17. The method of any one of claims 1-16, wherein the belumosudil is administered in a 28-day cycle.

18. The method of any one of claims 1-17, wherein the belumosudil is administered to the subject at a dose of up to 1000 mg per day.

19. The method of claim 18, wherein the dose is 200 mg per day.

20. The method of claim 18, wherein the dose is 200 mg twice per day.

21. The method of claim 18, wherein the dose is 400 mg per day.

22. The method of claim 18, wherein the dose is selected from 500 mg per day, 600 mg per day, 700 mg per day, 800 mg per day, 900 mg per day and 1000 mg per day.

23. The method of any one of claims 1-22, wherein the belumosudil is administered orally.

24. The method of any one of claims 1-23, wherein belumosudil is the mesylate salt of 2-{3-[4-(1H-indazol-5-ylamino)-2-quinazolinyl]phenoxy}-N-(propan-2-yl) acetamide.

25. The method of any one of claims 1-24, wherein the subject is receiving concomitant corticosteroid therapy.

26. The method of claim 25, wherein the concomitant corticosteroid therapy is selected from dexamethasone, prednisone, and methylprednisolone.

27. The method of claim 1, further comprising administering isatuximab and dexamethasone.

28. The method of claim 1, further comprising administering pomalidomide and dexamethasone.

29. The method of any one of claims 1-28, wherein the subject has received prior therapy for treating the multiple myeloma.

30. The method of claim 29, wherein the subject has relapsed multiple myeloma.

31. The method of claim 28 or 29, wherein the subject has relapsed and refractory multiple myeloma.

32. The method of any one of claims 1-29, wherein the multiple myeloma is smoldering multiple myeloma.

33. The method of any one of claims 1-32, wherein the treatment overcomes IMiD resistance in the subject with multiple myeloma.
